(19) United States
(12) Patent Application Publication
(10) Pub. No.: US 2022/0162590 A1
(43) Pub. Date: May 26, 2022

(54) METHODS FOR ACCURATE BASE CALLING USING MOLECULAR BARCODES

(71) Applicant: Ultima Genomics, Inc., Newark, CA (US)

(72) Inventors: Gilad ALMOGY, Palo Alto, CA (US); Eyal NEISTEIN, Herzliya (IL); Mark PRATT, Bozeman, MT (US)

(21) Appl. No.: 17/546,978

(22) Filed: Dec. 9, 2021

Related U.S. Application Data
(63) Continuation of application No. PCT/US2020/037595, filed on Jun. 12, 2020.
(60) Provisional application No. 62/860,462, filed on Jun. 12, 2019.

(51) Int. Cl. C12N 15/10; G16B 20/20; G16B 40/20; C12Q 1/6869

(52) U.S. Cl. CPC C12N 15/1065; C12Q 1/6869; G16B 40/20; G16B 20/20

(57) ABSTRACT

The present disclosure provides methods for accurate base calling of sequences using molecular barcodes. A method for sequencing nucleic acid molecules may comprise: (a) using barcode molecules to barcode nucleic acid molecules from a sample, to generate barcoded nucleic acid molecules comprising barcode sequences; (b) sequencing the barcoded nucleic acid molecules to generate sequencing signals comprising signals corresponding to the barcode sequences, wherein the sequencing signals are not sequencing reads; (c) using the signals corresponding to the barcode sequences to group the sequencing signals into groups, wherein sequencing signals of a given group comprise signals corresponding to a barcode sequence that is (i) identical for the given group and (ii) different from barcode sequences of other groups; (d) processing the sequencing signals within the given group to generate sets of aggregated signals which are not sequencing reads; and (e) combining the sets of aggregated signals to generate a consensus sequence.

### CROSS-REFERENCE

This application is a continuation of International Patent Application No. PCT/US2020/037595, filed on Jun. 12, 2020, claims the benefit of U.S. Provisional Patent Application No. 62/860,462, filed Jun. 12, 2019, which is incorporated by reference herein in its entirety.

### BACKGROUND

The goal to elucidate the entire human genome has created interest in technologies for rapid nucleic acid (e.g., deoxyribonucleic acid (DNA) or ribonucleic acid (RNA)) sequencing, both for small and large scale applications. As knowledge of the genetic basis for human diseases increases, high-throughput DNA sequencing has been leveraged for myriad clinical applications. Despite the prevalence of nucleic acid sequencing methods and systems in a wide range of molecular biology and diagnostics applications, such methods and systems may encounter challenges in accurate base calling. In particular, sequencing methods that perform base calling based on quantified characteristic signals indicating nucleotide incorporation can have sequencing errors, stemming from fundamental random errors (e.g., Poisson noise in detection and binomial noise from biochemistry processes) and/or unpredictable systematic variations in signal levels and context dependent signals that may be different for every sequence. Such signal variations and context dependency signals may cause issues with sequence calling.

### SUMMARY

Recognized herein is a need for improved base calling of sequences. Methods and systems provided herein can significantly reduce or eliminate errors in base calling and/or homopolymer length assessment of sequences resulting from fundamental random errors (e.g., Poisson noise in detection and binomial noise from biochemistry processes), which can generally be reduced by the square root of the number of replicates. Methods and systems of the present disclosure may use molecular barcodes to group sequencing signals, aggregate sequencing signals within groups, and combining aggregated sequencing signals to generate consensus sequences. Such methods and systems may achieve accurate and efficient base calling of sequences with very low single-copy error rates, which are required to maximize sensitivity of detecting rare events while maximizing specificity (e.g., minimizing false detections).

In an aspect, the present disclosure provides a method for sequencing a plurality of nucleic acid molecules, comprising: (a) using a plurality of barcode molecules to barcode a plurality of nucleic acid molecules from a biological sample, to generate a plurality of barcoded nucleic acid molecules comprising a plurality of barcode sequences; (b) sequencing the plurality of barcoded nucleic acid molecules to generate a plurality of sequencing signals, which plurality of sequencing signals comprises signals corresponding to the plurality of barcode sequences, wherein the plurality of sequencing signals are not sequencing reads; (c) using the signals corresponding to the plurality of barcode sequences to group the plurality of sequencing signals into a plurality of groups, wherein sequencing signals of a given group of the plurality of groups comprise signals corresponding to a barcode sequence of the plurality of barcode sequences that is (i) identical for the given group and (ii) different from barcode sequences of other groups of the plurality of groups; (d) processing the sequencing signals within the given group to generate one or more sets of aggregated signals, wherein the one or more sets of aggregated signals are not sequencing reads; and (e) combining the one or more sets of aggregated signals to generate a consensus sequence.

In some embodiments, in (e), the combining comprises performing base calling to identify individual bases. In some embodiments, the base calling is performed by processing aggregated signals within each of the one or more sets of aggregated signals to each other to generate the consensus sequence. In some embodiments, the method further comprises averaging the aggregated signals within each of the one or more sets of aggregated signals to each other to generate the consensus sequence. In some embodiments, the method further comprises processing the consensus sequence against a reference to identify one or more genetic variants. In some embodiments, the base calling is performed by processing aggregated signals within each of the one or more sets of aggregated signals against a reference signal to generate the consensus sequence. In some embodiments, the plurality of nucleic acid molecules is obtained from a bodily sample of a subject. In some embodiments, the plurality of nucleic acid molecules comprises deoxyribonucleic acid (DNA) molecules. In some embodiments, the DNA molecules comprise methylated DNA molecules. In some embodiments, the plurality of nucleic acid molecules comprises ribonucleic acid (RNA) molecules. In some embodiments, in (a), the barcoding comprises ligating the barcode molecules to the plurality of nucleic acid molecules. In some embodiments, the plurality of barcoded nucleic acid molecules is non-uniquely barcoded. In some embodiments, the plurality of barcode molecules comprises at least about 100,000 distinct barcodes. In some embodiments, the plurality of barcode molecules comprises a Hamming distance of at least 2 nucleotide substitutions. In some embodiments, the plurality of sequencing signals comprises analog signals. In some embodiments, the method further comprises, prior to or after (c), pre-processing the plurality of sequencing signals to remove systematic errors. In some embodiments, the method further comprises, prior to (b), amplifying the plurality of barcoded nucleic acid molecules. In some embodiments, the amplifying comprises polymerase chain reaction (PCR). In some embodiments, the amplifying comprises recombinase polymerase amplification (RPA). In some embodiments, the plurality of sequencing signals is generated by massively parallel array sequencing. In some embodiments, the plurality of sequencing signals is generated by flow sequencing. In some embodiments, (c) and (d) are performed in real time or near real time with the sequencing of (b). In some embodiments, (e) is performed in real time or near real time with the sequencing of (b).

In an aspect, the present disclosure provides a system for sequencing a plurality of nucleic acid molecules, comprising: a database that stores a plurality of sequencing signals generated upon using a plurality of barcode molecules to barcode the plurality of nucleic acid molecules and sequencing the plurality of barcoded nucleic acid molecules, which plurality of sequencing signals comprises signals corresponding to the plurality of barcode sequences, wherein the plurality of sequencing signals are not sequencing reads; and one or more computer processors operatively coupled to the database, wherein the one or more computer processors are individually or collectively programmed to: use the signals corresponding to the plurality of barcode sequences to group the plurality of sequencing signals into a plurality of groups, wherein sequencing signals of a given group of the plurality of groups comprise signals corresponding to a barcode sequence of the plurality of barcode sequences that is (i) identical for the given group and (ii) different from barcode sequences of other groups of the plurality of groups; process the sequencing signals within the given group to generate one or more sets of aggregated signals, wherein the one or more sets of aggregated signals are not sequencing reads; and combine the one or more sets of aggregated signals to generate a consensus sequence.

In another aspect, the present disclosure provides a method for sequencing a plurality of nucleic acid molecules, comprising: (a) using a plurality of barcode molecules to barcode a plurality of nucleic acid molecules from a biological sample, to generate a plurality of barcoded nucleic acid molecules comprising a plurality of barcode sequences; (b) sequencing the plurality of barcoded nucleic acid molecules to generate a plurality of sequencing signals, which plurality of sequencing signals comprises signals corresponding to the plurality of barcode sequences, wherein the plurality of sequencing signals are not sequencing reads; (c) processing the signals corresponding to the plurality of barcode sequences to identify the barcode sequences of each of the plurality of sequencing signals; (d) using the identified barcode sequences to group the plurality of sequencing signals into a plurality of groups, wherein sequencing signals of a given group of the plurality of groups correspond to an identified barcode sequence of the plurality of barcode sequences that is (i) identical for the given group and (ii) different from identified barcode sequences of other groups of the plurality of groups; (e) processing the sequencing signals within the given group to generate one or more sets of aggregated signals, wherein the one or more sets of aggregated signals are not sequencing reads; and (f) combining the one or more sets of aggregated signals to generate a consensus sequence.

In some embodiments, in (f), the combining comprises performing base calling to identify individual bases. In some embodiments, the base calling is performed by processing aggregated signals within each of the one or more sets of aggregated signals to each other to generate the consensus sequence. In some embodiments, the processing comprises averaging the aggregated signals within each of the one or more sets of aggregated signals to each other to generate the consensus sequence. In some embodiments, the method further comprises processing the consensus sequence against a reference to identify one or more genetic variants. In some embodiments, the base calling is performed by processing aggregated signals within each of the one or more sets of aggregated signals against a reference signal to generate the consensus sequence. In some embodiments, the plurality of nucleic acid molecules is obtained from a bodily sample of a subject. In some embodiments, the plurality of nucleic acid molecules comprises deoxyribonucleic acid (DNA) molecules. In some embodiments, the DNA molecules comprise methylated DNA molecules. In some embodiments, the plurality of nucleic acid molecules comprises ribonucleic acid (RNA) molecules. In some embodiments, in (a), the barcoding comprises ligating the barcode molecules to the plurality of nucleic acid molecules. In some embodiments, the plurality of barcoded nucleic acid molecules is non-uniquely barcoded. In some embodiments, the plurality of barcode molecules comprises at least about 100 thousand distinct barcodes. In some embodiments, the plurality of barcode molecules comprises a Hamming distance of at least 2 nucleotide substitutions. In some embodiments, the plurality of sequencing signals comprises analog signals. In some embodiments, the method further comprises, prior to or after (d), pre-processing the plurality of sequencing signals to remove systematic errors. In some embodiments, the method further comprises, prior to (b), amplifying the plurality of barcoded nucleic acid molecules. In some embodiments, the amplifying comprises polymerase chain reaction (PCR). In some embodiments, the amplifying comprises recombinase polymerase amplification (RPA). In some embodiments, the plurality of sequencing signals is generated by massively parallel array sequencing. In some embodiments, the plurality of sequencing signals is generated by flow sequencing. In some embodiments, (d) and (e) are performed in real time or near real time with the sequencing of (b). In some embodiments, (f) is performed in real time or near real time with the sequencing of (b).

In another aspect, the present disclosure provides a system for sequencing a plurality of nucleic acid molecules, comprising: a database that stores a plurality of sequencing signals generated upon using a plurality of barcode molecules to barcode the plurality of nucleic acid molecules and sequencing the plurality of barcoded nucleic acid molecules, which plurality of sequencing signals comprises signals corresponding to the plurality of barcode sequences, wherein the plurality of sequencing signals are not sequencing reads; and one or more computer processors operatively coupled to the database, wherein the one or more computer processors are individually or collectively programmed to: process the signals corresponding to the plurality of barcode sequences to identify the barcode sequences of each of the plurality of sequencing signals; use the identified barcode sequences to group the plurality of sequencing signals into a plurality of groups, wherein sequencing signals of a given group of the plurality of groups correspond to an identified barcode sequence of the plurality of barcode sequences that is (i) identical for the given group and (ii) different from identified barcode sequences of other groups of the plurality of groups; process the sequencing signals within the given group to generate one or more sets of aggregated signals, wherein the one or more sets of aggregated signals are not sequencing reads; and combine the one or more sets of aggregated signals to generate a consensus sequence.

In another aspect, the present disclosure provides a method for sequencing a plurality of nucleic acid molecules, comprising: (a) using a plurality of barcode molecules to barcode a plurality of nucleic acid molecules from a biological sample, to generate a plurality of barcoded nucleic acid molecules comprising a plurality of barcode sequences; (b) sequencing the plurality of barcoded nucleic acid molecules to generate a plurality of sequencing signals, which plurality of sequencing signals comprises signals corresponding to the plurality of barcode sequences, wherein the plurality of sequencing signals are not sequencing reads; (c) using the signals corresponding to the plurality of barcode sequences to group the plurality of sequencing signals into a plurality of groups, wherein sequencing signals of a given group of the plurality of groups comprise signals corresponding to a barcode sequence of the plurality of barcode sequences that is (i) identical for the given group and (ii) different from barcode sequences of other groups of the plurality of groups; (d) processing the sequencing signals within the given group to generate one or more estimated sequences, wherein each of the one or more estimated sequences comprises a plurality of estimated base calls; and (e) combining the one or more estimated sequences to generate a consensus sequence.

In some embodiments, the one or more estimated sequences comprise a plurality of estimated sequences, and the consensus sequence is generated based on a majority vote among the plurality of estimated sequences. In some embodiments, the method further comprises processing the consensus sequence against a reference to identify one or more genetic variants. In some embodiments, the plurality of nucleic acid molecules is obtained from a bodily sample of a subject. In some embodiments, the plurality of nucleic acid molecules comprises deoxyribonucleic acid (DNA) molecules. In some embodiments, the DNA molecules comprise methylated DNA molecules. In some embodiments, the plurality of nucleic acid molecules comprises ribonucleic acid (RNA) molecules. In some embodiments, in (a), the barcoding comprises ligating the barcode molecules to the plurality of nucleic acid molecules. In some embodiments, the plurality of barcoded nucleic acid molecules is non-uniquely barcoded. In some embodiments, the plurality of barcode molecules comprises at least about 100 thousand distinct barcodes. In some embodiments, the plurality of barcode molecules comprises a Hamming distance of at least 2 nucleotide substitutions. In some embodiments, the plurality of sequencing signals comprises analog signals. In some embodiments, the method further comprises, prior to or after (c), pre-processing the plurality of sequencing signals to remove systematic errors. In some embodiments, the method further comprises, prior to (b), amplifying the plurality of barcoded nucleic acid molecules. In some embodiments, the amplifying comprises polymerase chain reaction (PCR). In some embodiments, the amplifying comprises recombinase polymerase amplification (RPA). In some embodiments, the plurality of sequencing signals is generated by massively parallel array sequencing. In some embodiments, the plurality of sequencing signals is generated by flow sequencing. In some embodiments, (c) and (d) are performed in real time or near real time with the sequencing of (b). In some embodiments, (e) is performed in real time or near real time with the sequencing of (b).

In another aspect, the present disclosure provides a system for sequencing a plurality of nucleic acid molecules, comprising: a database that stores a plurality of sequencing signals generated upon using a plurality of barcode molecules to barcode the plurality of nucleic acid molecules and sequencing the plurality of barcoded nucleic acid molecules, which plurality of sequencing signals comprises signals corresponding to the plurality of barcode sequences, wherein the plurality of sequencing signals are not sequencing reads; and one or more computer processors operatively coupled to the database, wherein the one or more computer processors are individually or collectively programmed to: use the signals corresponding to the plurality of barcode sequences to group the plurality of sequencing signals into a plurality of groups, wherein sequencing signals of a given group of the plurality of groups comprise signals corresponding to a barcode sequence of the plurality of barcode sequences that is (i) identical for the given group and (ii) different from barcode sequences of other groups of the plurality of groups; process the sequencing signals within the given group to generate one or more estimated sequences, wherein each of the one or more estimated sequences comprises a plurality of estimated base calls; and combine the one or more estimated sequences to generate a consensus sequence.

In another aspect, the present disclosure provides a method for sequencing a plurality of nucleic acid molecules, comprising: (a) using a plurality of barcode molecules to barcode a plurality of nucleic acid molecules from a biological sample, to generate a plurality of barcoded nucleic acid molecules comprising a plurality of barcode sequences; (b) sequencing the plurality of barcoded nucleic acid molecules to generate a plurality of sequencing signals, which plurality of sequencing signals comprises signals corresponding to the plurality of barcode sequences, wherein the plurality of sequencing signals are not sequencing reads; (c) processing the signals corresponding to the plurality of barcode sequences to identify the barcode sequences of each of the plurality of sequencing signals; (d) using the identified barcode sequences to group the plurality of sequencing signals into a plurality of groups, wherein sequencing signals of a given group of the plurality of groups correspond to an identified barcode sequence of the plurality of barcode sequences that is (i) identical for the given group and (ii) different from barcode sequences of other groups of the plurality of groups; (e) processing the sequencing signals within the given group to generate one or more estimated sequences, wherein each of the one or more estimated sequences comprises a plurality of estimated base calls; and (f) combining the one or more estimated sequences to generate a consensus sequence.

In some embodiments, the one or more estimated sequences comprise a plurality of estimated sequences, and the consensus sequence is generated based on a majority vote among the plurality of estimated sequences. In some embodiments, the method further comprises processing the consensus sequence against a reference to identify one or more genetic variants. In some embodiments, the plurality of nucleic acid molecules is obtained from a bodily sample of a subject. In some embodiments, the plurality of nucleic acid molecules comprises deoxyribonucleic acid (DNA) molecules. In some embodiments, the DNA molecules comprise methylated DNA molecules. In some embodiments, the plurality of nucleic acid molecules comprises ribonucleic acid (RNA) molecules. In some embodiments, in (a), the barcoding comprises ligating the barcode molecules to the plurality of nucleic acid molecules. In some embodiments, the plurality of barcoded nucleic acid molecules is non-uniquely barcoded. In some embodiments, the plurality of barcode molecules comprises at least about 100 thousand distinct barcodes. In some embodiments, the plurality of barcode molecules comprises a Hamming distance of at least 2 nucleotide substitutions. In some embodiments, the plurality of sequencing signals comprises analog signals. In some embodiments, the method further comprises, prior to or after (d), pre-processing the plurality of sequencing signals to remove systematic errors. In some embodiments, the method further comprises, prior to (b), amplifying the plurality of barcoded nucleic acid molecules. In some embodiments, the amplifying comprises polymerase chain reaction (PCR). In some embodiments, the amplifying comprises recombinase polymerase amplification (RPA). In some embodiments, the plurality of sequencing signals is generated by massively parallel array sequencing. In some embodiments, the plurality of sequencing signals is generated by flow sequencing. In some embodiments, (d) and (e) are performed in real time or near real time with the sequencing of (b). In some embodiments, (f) is performed in real time or near real time with the sequencing of (b).

In another aspect, the present disclosure provides a system for sequencing a plurality of nucleic acid molecules, comprising: a database that stores a plurality of sequencing signals generated upon using a plurality of barcode molecules to barcode the plurality of nucleic acid molecules and sequencing the plurality of barcoded nucleic acid molecules, which plurality of sequencing signals comprises signals corresponding to the plurality of barcode sequences, wherein the plurality of sequencing signals are not sequencing reads; and one or more computer processors operatively coupled to the database, wherein the one or more computer processors are individually or collectively programmed to: process the signals corresponding to the plurality of barcode sequences to identify the barcode sequences of each of the plurality of sequencing signals; use the identified barcode sequences to group the plurality of sequencing signals into a plurality of groups, wherein sequencing signals of a given group of the plurality of groups correspond to an identified barcode sequence of the plurality of barcode sequences that is (i) identical for the given group and (ii) different from identified barcode sequences of other groups of the plurality of groups; process the sequencing signals within the given group to generate one or more estimated sequences, wherein each of the one or more estimated sequences comprises a plurality of estimated base calls; and combine the one or more estimated sequences to generate a consensus sequence.

Additional aspects and advantages of the present disclosure will become readily apparent to those skilled in this art from the following detailed description, wherein only illustrative embodiments of the present disclosure are shown and described. As will be realized, the present disclosure is capable of other and different embodiments, and its several details are capable of modifications in various obvious respects, all without departing from the disclosure. Accordingly, the drawings and description are to be regarded as illustrative in nature, and not as restrictive.

### INCORPORATION BY REFERENCE

All publications, patents, and patent applications mentioned in this specification are herein incorporated by reference to the same extent as if each individual publication, patent, or patent application was specifically and individually indicated to be incorporated by reference. To the extent publications and patents or patent applications incorporated by reference contradict the disclosure contained in the specification, the specification is intended to supersede and/or take precedence over any such contradictory material.

### BRIEF DESCRIPTION OF THE DRAWINGS

The novel features of the invention are set forth with particularity in the appended claims. A better understanding of the features and advantages of the present invention will be obtained by reference to the following detailed description that sets forth illustrative embodiments, in which the principles of the invention are utilized, and the accompanying drawings (also “Figure” and “FIG.” herein), of which:

FIG. 1 shows an example of a flowchart illustrating methods of base calling using molecular barcodes, in accordance with disclosed embodiments.

FIG. 2 shows an example of a plurality of amplified barcoded library fragment signal reads, in accordance with disclosed embodiments.

FIG. 3 shows an example of a plurality of amplified barcoded library fragment signal reads, which have been classified based on their barcodes and grouped into smaller barcode-specific pools, in accordance with disclosed embodiments.

FIG. 4 shows an example of performing a read-read alignment within each barcode pool, which provides template copy groups that can be analyzed to improve signal-to-noise ratio (SNR) and base call accuracy, thereby allowing rare variant calls based on single input copies, in accordance with disclosed embodiments.

FIG. 5 shows a computer system that is programmed or otherwise configured to implement methods provided herein.

FIG. 6 shows an example of data generated using flow signals for a TF1L template and a human genome-trained neural network model for base calling.

FIG. 7 shows an example of data generated using flow signals for a TF4L template and a human genome-trained neural network model for base calling.

FIG. 8 shows an example of data generated using flow signals for a TF3L template and an E. coli genome-trained neural network model for base calling.

FIG. 9 shows an example of data generated using flow signals for a TF4L template and an E. coli genome-trained neural network model for base calling.

### DETAILED DESCRIPTION

While various embodiments of the invention have been shown and described herein, it will be obvious to those skilled in the art that such embodiments are provided by way of example only. Numerous variations, changes, and substitutions may occur to those skilled in the art without departing from the invention. It should be understood that various alternatives to the embodiments of the invention described herein may be employed.

The term “sequencing,” as used herein, generally refers to a process for generating or identifying a sequence of a biological molecule, such as a nucleic acid molecule. Such sequence may be a nucleic acid sequence, which may include a sequence of nucleic acid bases. Sequencing methods may be massively parallel array sequencing (e.g., Illumina sequencing), which may be performed using template nucleic acid molecules immobilized on a support, such as a flow cell or beads. Sequencing methods may include, but are not limited to: high-throughput sequencing, next-generation sequencing, sequencing-by-synthesis, flow sequencing, massively-parallel sequencing, shotgun sequencing, single-molecule sequencing, nanopore sequencing, pyrosequencing, semiconductor sequencing, sequencing-by-ligation, sequencing-by-hybridization, ribonucleic acid (RNA) sequencing (RNA-Seq) (Illumina), Digital Gene Expression (Helicos), Single Molecule Sequencing by Synthesis (SMSS) (Helicos), Clonal Single Molecule Array (Solexa), and Maxim-Gilbert sequencing.

The term “flow sequencing,” as used herein, generally refers to a sequencing-by-synthesis (SBS) process in which cyclic or acyclic introduction of single nucleotide solutions produce discrete deoxyribonucleic acid (DNA) extensions that are sensed (e.g., by a detector that detects fluorescence signals from the DNA extensions).

The term “subject,” as used herein, generally refers to an individual having a biological sample that is undergoing processing or analysis. A subject can be an animal or plant. The subject can be a mammal, such as a human, dog, cat, horse, pig, or rodent. The subject can have or be suspected of having a disease, such as cancer (e.g., breast cancer, colorectal cancer, brain cancer, leukemia, lung cancer, skin cancer, liver cancer, pancreatic cancer, lymphoma, esophageal cancer or cervical cancer) or an infectious disease. The subject can have or be suspected of having a genetic disorder such as achondroplasia, alpha-1 antitrypsin deficiency, antiphospholipid syndrome, autism, autosomal dominant polycystic kidney disease, Charcot-Marie-tooth, cri du chat, Crohn's disease, cystic fibrosis, Dercum disease, down syndrome, Duane syndrome, Duchenne muscular dystrophy, factor V Leiden thrombophilia, familial hypercholesterolemia, familial Mediterranean fever, fragile x syndrome, Gaucher disease, hemochromatosis, hemophilia, holoprosencephaly, Huntington's disease, Klinefelter syndrome, Marfan syndrome, myotonic dystrophy, neurofibromatosis, Noonan syndrome, osteogenesis imperfecta, Parkinson's disease, phenylketonuria, Poland anomaly, porphyria, progeria, retinitis pigmentosa, severe combined immunodeficiency, sickle cell disease, spinal muscular atrophy, Tay-Sachs, thalassemia, trimethylaminuria, Turner syndrome, velocardiofacial syndrome, WAGR syndrome, or Wilson disease.

The term “sample,” as used herein, generally refers to a biological sample. Examples of biological samples include nucleic acid molecules, amino acids, polypeptides, proteins, carbohydrates, fats, or viruses. In an example, a biological sample is a nucleic acid sample including one or more nucleic acid molecules, such as deoxyribonucleic acid (DNA) and/or ribonucleic acid (RNA). The nucleic acid molecules may be cell-free or cell-free nucleic acid molecules, such as cell-free DNA or cell-free RNA. The nucleic acid molecules may be derived from a variety of sources including human, mammal, non-human mammal, ape, monkey, chimpanzee, reptilian, amphibian, or avian, sources. Further, samples may be extracted from variety of animal fluids containing cell free sequences, including but not limited to blood, serum, plasma, vitreous, sputum, urine, tears, perspiration, saliva, semen, mucosal excretions, mucus, spinal fluid, amniotic fluid, lymph fluid and the like. Cell free polynucleotides may be fetal in origin (via fluid taken from a pregnant subject), or may be derived from tissue of the subject itself.

The term “nucleic acid,” or “polynucleotide,” as used herein, generally refers to a molecule comprising one or more nucleic acid subunits, or nucleotides. A nucleic acid may include one or more nucleotides selected from adenosine (A), cytosine (C), guanine (G), thymine (T) and uracil (U), or variants thereof. A nucleotide generally includes a nucleoside and at least 1, 2, 3, 4, 5, 6, 7, 8, 9, 10, or more phosphate (PO₃) groups. A nucleotide can include a nucleobase, a five-carbon sugar (either ribose or deoxyribose), and one or more phosphate groups.

Ribonucleotides are nucleotides in which the sugar is ribose. Deoxyribonucleotides are nucleotides in which the sugar is deoxyribose. A nucleotide can be a nucleoside monophosphate or a nucleoside polyphosphate. A nucleotide can be a deoxyribonucleoside polyphosphate, such as, e.g., a deoxyribonucleoside triphosphate (dNTP), which can be selected from deoxyadenosine triphosphate (dATP), deoxycytidine triphosphate (dCTP), deoxyguanosine triphosphate (dGTP), uridine triphosphate (dUTP) and deoxythymidine triphosphate (dTTP) dNTPs, that include detectable tags, such as luminescent tags or markers (e.g., fluorophores). A nucleotide can include any subunit that can be incorporated into a growing nucleic acid strand. Such subunit can be an A, C, G, T, or U, or any other subunit that is specific to one or more complementary A, C, G, T or U, or complementary to a purine (i.e., A or G, or variant thereof) or a pyrimidine (i.e., C, T or U, or variant thereof). In some examples, a nucleic acid is deoxyribonucleic acid (DNA), ribonucleic acid (RNA), or derivatives or variants thereof. A nucleic acid may be single-stranded or double-stranded. In some cases, a nucleic acid molecule is circular.

The terms “nucleic acid molecule,” “nucleic acid sequence,” “nucleic acid fragment,” “oligonucleotide” and “polynucleotide,” as used herein, generally refer to a polynucleotide that may have various lengths, such as either deoxyribonucleotides or ribonucleotides (RNA), or analogs thereof. A nucleic acid molecule can have a length of at least about 10 bases, 20 bases, 30 bases, 40 bases, 50 bases, 100 bases, 200 bases, 300 bases, 400 bases, 500 bases, 1 kilobase (kb), 2 kb, 3 kb, 4 kb, 5 kb, 10 kb, 50 kb, or more. An oligonucleotide is typically composed of a specific sequence of four nucleotide bases: adenine (A); cytosine (C); guanine (G); and thymine (T) (uracil (U) for thymine (T) when the polynucleotide is RNA). Thus, the term “oligonucleotide sequence” is the alphabetical representation of a polynucleotide molecule; alternatively, the term may be applied to the polynucleotide molecule itself. This alphabetical representation can be input into databases in a computer having a central processing unit and used for bio informatics applications such as functional genomics and homology searching. Oligonucleotides may include one or more nonstandard nucleotide(s), nucleotide analog(s), and/or modified nucleotides.

The term “nucleotide analogs,” as used herein, may include, but are not limited to, diaminopurine, 5-fluorouracil, 5-bromouracil, 5-chlorouracil, 5-iodouracil, hypoxanthine, xantine, 4-acetylcytosine, 5-(carboxyhydroxylmethyl)uracil, 5-carboxymethylaminomethyl-2-thiouridine, 5-carboxymethylaminomethyluracil, dihydrouracil, beta-D-galactosylqueosine, inosine, N6-isopentenyladenine, 1-methylguanine, 1-methylinosine, 2,2-dimethylguanine, 2-methyladenine, 2-methylguanine, 3-methylcytosine, 5-methylcytosine, N6-adenine, 7-methylguanine, 5-methylaminomethyluracil, 5-methoxyaminomethyl-2-thiouracil, beta-D-mannosylqueosine, 5′-methoxycarboxymethyluracil, 5-methoxyuracil, 2-methylthio-D46-isopentenyladenine, uracil-5-oxyacetic acid (v), wybutoxosine, pseudouracil, queosine, 2-thiocytosine, 5-methyl-2-thiouracil, 2-thiouracil, 4-thiouracil, 5-methyluracil, uracil-5-oxyacetic acid methylester, uracil-5-oxyacetic acid(v), 5-methyl-2-thiouracil, 3-(3-amino-3-N-2-carboxypropyl) uracil, (acp3)w, 2,6-diaminopurine, phosphoroselenoate nucleic acids, and the like. In some cases, nucleotides may include modifications in their phosphate moieties, including modifications to a triphosphate moiety. Additional, non-limiting examples of modifications include phosphate chains of greater length (e.g., a phosphate chain having 4, 5, 6, 7, 8, 9, 10, or more than 10 phosphate moieties), modifications with thiol moieties (e.g., alpha-thio triphosphate and beta-thiotriphosphates) or modifications with selenium moieties (e.g., phosphoroselenoate nucleic acids). Nucleic acid molecules may also be modified at the base moiety (e.g., at one or more atoms that typically are available to form a hydrogen bond with a complementary nucleotide and/or at one or more atoms that are not typically capable of forming a hydrogen bond with a complementary nucleotide), sugar moiety or phosphate backbone. Nucleic acid molecules may also contain amine-modified groups, such as aminoallyl-dUTP (aa-dUTP) and aminohexhylacrylamide-dCTP (aha-dCTP) to allow covalent attachment of amine reactive moieties, such as N-hydroxysuccinimide esters (NHS). Alternatives to standard DNA base pairs or RNA base pairs in the oligonucleotides of the present disclosure can provide higher density in bits per cubic millimeter (mm), higher safety (e.g., resistance to accidental or purposeful synthesis of natural toxins), easier discrimination in photo-programmed polymerases, or lower secondary structure. Nucleotide analogs may be capable of reacting or bonding with detectable moieties for nucleotide detection.

The term “free nucleotide analog” as used herein, generally refers to a nucleotide analog that is not coupled to an additional nucleotide or nucleotide analog. Free nucleotide analogs may be incorporated in to the growing nucleic acid chain by primer extension reactions.

The term “primer(s),” as used herein, generally refers to a polynucleotide which is complementary to the template nucleic acid. The complementarity or homology or sequence identity between the primer and the template nucleic acid may be limited. The length of the primer may be between 8 nucleotide bases to 50 nucleotide bases. The length of the primer may be greater than or equal to 6 nucleotide bases, 7 nucleotide bases, 8 nucleotide bases, 9 nucleotide bases, 10 nucleotide bases, 11 nucleotide bases, 12 nucleotide bases, 13 nucleotide bases, 14 nucleotide bases, 15 nucleotide bases, 16 nucleotide bases, 17 nucleotide bases, 18 nucleotide bases, 19 nucleotide bases, 20 nucleotide bases, 21 nucleotide bases, 22 nucleotide bases, 23 nucleotide bases, 24 nucleotide bases, 25 nucleotide bases, 26 nucleotide bases, 27 nucleotide bases, 28 nucleotide bases, 29 nucleotide bases, 30 nucleotide bases, 31 nucleotide bases, 32 nucleotide bases, 33 nucleotide bases, 34 nucleotide bases, 35 nucleotide bases, 37 nucleotide bases, 40 nucleotide bases, 42 nucleotide bases, 45 nucleotide bases, 47 nucleotide bases, or 50 nucleotide bases.

A primer may exhibit sequence identity or homology or complementarity to the template nucleic acid. The homology or sequence identity or complementarity between the primer and a template nucleic acid may be based on the length of the primer. For example, if the primer length is about 20 nucleic acids, it may contain 10 or more contiguous nucleic acid bases complementary to the template nucleic acid.

The term “primer extension reaction,” as used herein, generally refers to the binding of a primer to a strand of the template nucleic acid, followed by elongation of the primer(s). It may also include, denaturing of a double-stranded nucleic acid and the binding of a primer strand to either one or both of the denatured template nucleic acid strands, followed by elongation of the primer(s). Primer extension reactions may be used to incorporate nucleotides or nucleotide analogs to a primer in template-directed fashion by using enzymes (polymerizing enzymes).

The term “polymerase,” as used herein, generally refers to any enzyme capable of catalyzing a polymerization reaction. Examples of polymerases include, without limitation, a nucleic acid polymerase. The polymerase can be naturally occurring or synthesized. In some cases, a polymerase has relatively high processivity. An example polymerase is a Φ29 polymerase or a derivative thereof. A polymerase can be a polymerization enzyme. In some cases, a transcriptase or a ligase is used (i.e., enzymes which catalyze the formation of a bond).

Examples of polymerases include a DNA polymerase, an RNA polymerase, a thermostable polymerase, a wild-type polymerase, a modified polymerase, E. coli DNA polymerase I, T7 DNA polymerase, bacteriophage T4 DNA polymerase Φ29 (phi29) DNA polymerase, Taq polymerase, Tth polymerase, Tli polymerase, Pfu polymerase, Pwo polymerase, VENT polymerase, DEEPVENT polymerase, EX-Taq polymerase, LA-Taq polymerase, Sso polymerase, Poc polymerase, Pab polymerase, Mth polymerase, ES4 polymerase, Tru polymerase, Tac polymerase, Tne polymerase, Tma polymerase, Tea polymerase, Tih polymerase, Tfi polymerase, Platinum Taq polymerases, Tbr polymerase, Tfl polymerase, Pfutubo polymerase, Pyrobest polymerase, Pwo polymerase, KOD polymerase, Bst polymerase, Sac polymerase, Klenow fragment, polymerase with 3′ to 5′ exonuclease activity, and variants, modified products and derivatives thereof. In some cases, the polymerase is a single subunit polymerase. The polymerase can have high processivity, namely the capability of the polymerase to consecutively incorporate nucleotides into a nucleic acid template without releasing the nucleic acid template. In some cases, a polymerase is a polymerase modified to accept dideoxynucleotide triphosphates, such as for example, Taq polymerase having a 667Y mutation (see e.g., Tabor et al, PNAS, 1995, 92, 6339-6343, which is herein incorporated by reference in its entirety for all purposes). In some cases, a polymerase is a polymerase having a modified nucleotide binding, which may be useful for nucleic acid sequencing, with non-limiting examples that include ThermoSequenas polymerase (GE Life Sciences), AmpliTaq FS (ThermoFisher) polymerase and Sequencing Pol polymerase (Jena Bioscience). In some cases, the polymerase is genetically engineered to have discrimination against dideoxynucleotides, such, as for example, Sequenase DNA polymerase (ThermoFisher).

The term “support,” as used herein, generally refers to a solid support such as a slide, a bead, a resin, a chip, an array, a matrix, a membrane, a nanopore, or a gel. The solid support may, for example, be a bead on a flat substrate (such as glass, plastic, silicon, etc.) or a bead within a well of a substrate. The substrate may have surface properties, such as textures, patterns, microstructure coatings, surfactants, or any combination thereof to retain the bead at a desire location (such as in a position to be in operative communication with a detector). The detector of bead-based supports may be configured to maintain substantially the same read rate independent of the size of the bead. The support may be a flow cell or an open substrate. Furthermore, the support may comprise a biological support, a non-biological support, an organic support, an inorganic support, or any combination thereof. The support may be in optical communication with the detector, may be physically in contact with the detector, may be separated from the detector by a distance, or any combination thereof. The support may have a plurality of independently addressable locations. The nucleic acid molecules may be immobilized to the support at a given independently addressable location of the plurality of independently addressable locations. Immobilization of each of the plurality of nucleic acid molecules to the support may be aided by the use of an adaptor. The support may be optically coupled to the detector. Immobilization on the support may be aided by an adaptor.

The term “label,” as used herein, generally refers to a moiety that is capable of coupling with a species, such as, for example, a nucleotide analog. In some cases, a label may be a detectable label that emits a signal (or reduces an already emitted signal) that can be detected. In some cases, such a signal may be indicative of incorporation of one or more nucleotides or nucleotide analogs. In some cases, a label may be coupled to a nucleotide or nucleotide analog, which nucleotide or nucleotide analog may be used in a primer extension reaction. In some cases, the label may be coupled to a nucleotide analog after the primer extension reaction. The label, in some cases, may be reactive specifically with a nucleotide or nucleotide analog. Coupling may be covalent or non-covalent (e.g., via ionic interactions, Van der Waals forces, etc.). In some cases, coupling may be via a linker, which may be cleavable, such as photo-cleavable (e.g., cleavable under ultra-violet light), chemically-cleavable (e.g., via a reducing agent, such as dithiothreitol (DTT), tris(2-carboxyethyl)phosphine (TCEP)) or enzymatically cleavable (e.g., via an esterase, lipase, peptidase, or protease).

In some cases, the label may be optically active. In some embodiments, an optically-active label is an optically-active dye (e.g., fluorescent dye). Non-limiting examples of dyes include SYBR green, SYBR blue, DAPI, propidium iodine, Hoeste, SYBR gold, ethidium bromide, acridines, proflavine, acridine orange, acriflavine, fluorcoumanin, ellipticine, daunomycin, chloroquine, distamycin D, chromomycin, homidium, mithramycin, ruthenium polypyridyls, anthramycin, phenanthridines and acridines, ethidium bromide, propidium iodide, hexidium iodide, dihydroethidium, ethidium homodimer-1 and -2, ethidium monoazide, and ACMA, Hoechst 33258, Hoechst 33342, Hoechst 34580, DAPI, acridine orange, 7-AAD, actinomycin D, LDS751, hydroxystilbamidine, SYTOX Blue, SYTOX Green, SYTOX Orange, POPO-1, POPO-3, YOYO-1, YOYO-3, TOTO-1, TOTO-3, JOJO-1, LOLO-1, BOBO-1, BOBO-3, PO-PRO-1, PO-PRO-3, BO-PRO-1, BO-PRO-3, TO-PRO-1, TO-PRO-3, TO-PRO-5, JO-PRO-1, LO-PRO-1, YO-PRO-1, YO-PRO-3, PicoGreen, OliGreen, RiboGreen, SYBR Gold, SYBR Green I, SYBR Green II, SYBR DX, SYTO-40, -41, -42, -43, -44, -45 (blue), SYTO-13, -16, -24, -21, -23, -12, -11, -20, -22, -15, -14, -25 (green), SYTO-81, -80, -82, -83, -84, -85 (orange), SYTO-64, -17, -59, -61, -62, -60, -63 (red), fluorescein, fluorescein isothiocyanate (FITC), tetramethyl rhodamine isothiocyanate (TRITC), rhodamine, tetramethyl rhodamine, R-phycoerythrin, Cy-2, Cy-3, Cy-3.5, Cy-5, Cy5.5, Cy-7, Texas Red, Phar-Red, allophycocyanin (APC), Sybr Green I, Sybr Green II, Sybr Gold, CellTracker Green, 7-AAD, ethidium homodimer I, ethidium homodimer II, ethidium homodimer III, ethidium bromide, umbelliferone, eosin, green fluorescent protein, erythrosin, coumarin, methyl coumarin, pyrene, malachite green, stilbene, lucifer yellow, cascade blue, dichlorotriazinylamine fluorescein, dansyl chloride, fluorescent lanthanide complexes such as those including europium and terbium, carboxy tetrachloro fluorescein, 5 and/or 6-carboxy fluorescein (FAM), VIC, 5- (or 6-) iodoacetamidofluorescein, 5-{[2(and 3)-5-(Acetylmercapto)-succinyl]amino} fluorescein (SAMSA-fluorescein), lissamine rhodamine B sulfonyl chloride, 5 and/or 6 carboxy rhodamine (ROX), 7-amino-methyl-coumarin, 7-Amino-4-methylcoumarin-3-acetic acid (AMCA), BODIPY fluorophores, 8-methoxypyrene-1,3,6-trisulfonic acid trisodium salt, 3,6-Disulfonate-4-amino-naphthalimide, phycobiliproteins, AlexaFluor 350, 405, 430, 488, 532, 546, 555, 568, 594, 610, 633, 635, 647, 660, 680, 700, 750, and 790 dyes, DyLight 350, 405, 488, 550, 594, 633, 650, 680, 755, and 800 dyes, or other fluorophores.

In some examples, labels may be nucleic acid intercalator dyes. Examples include, but are not limited to ethidium bromide, YOYO-1, SYBR Green, and EvaGreen. The near-field interactions between energy donors and energy acceptors, between intercalators and energy donors, or between intercalators and energy acceptors can result in the generation of unique signals or a change in the signal amplitude. For example, such interactions can result in quenching (i.e., energy transfer from donor to acceptor that results in non-radiative energy decay) or Forster resonance energy transfer (FRET) (i.e., energy transfer from the donor to an acceptor that results in radiative energy decay). Other examples of labels include electrochemical labels, electrostatic labels, colorimetric labels and mass tags.

The term “quencher,” as used herein, generally refers to molecules that can reduce an emitted signal. Labels may be quencher molecules. For example, a template nucleic acid molecule may be designed to emit a detectable signal. Incorporation of a nucleotide or nucleotide analog comprising a quencher can reduce or eliminate the signal, which reduction or elimination is then detected. In some cases, as described elsewhere herein, labeling with a quencher can occur after nucleotide or nucleotide analog incorporation. Examples of quenchers include Black Hole Quencher Dyes (Biosearch Technologies) such as BH1-0, BHQ-1, BHQ-3, BHQ-10); QSY Dye fluorescent quenchers (from Molecular Probes/Invitrogen) such QSY7, QSY9, QSY21, QSY35, and other quenchers such as Dabcyl and Dabsyl; Cy5Q and Cy7Q and Dark Cyanine dyes (GE Healthcare). Examples of donor molecules whose signals can be reduced or eliminated in conjunction with the above quenchers include fluorophores such as Cy3B, Cy3, or Cy5; Dy-Quenchers (Dyomics), such as DYQ-660 and DYQ-661; fluorescein-5-maleimide; 7-diethylamino-3-(4′-maleimidylphenyl)-4-methylcoumarin (CPM); N-(7-dimethylamino-4-methylcoumarin-3-yl) maleimide (DACM) and ATTO fluorescent quenchers (ATTO-TEC GmbH), such as ATTO 540Q, 580Q, 612Q, 647N, Atto-633-iodoacetamide, tetramethylrhodamine iodoacetamide or Atto-488 iodoacetamide. In some cases, the label may be a type that does not self-quench for example, Bimane derivatives such as Monobromobimane.

The term “detector,” as used herein, generally refers to a device that is capable of detecting a signal, including a signal indicative of the presence or absence of an incorporated nucleotide or nucleotide analog. In some cases, a detector can include optical and/or electronic components that can detect signals. The term “detector” may be used in detection methods. Non-limiting examples of detection methods include optical detection, spectroscopic detection, electrostatic detection, electrochemical detection, and the like. Optical detection methods include, but are not limited to, fluorimetry and UV-vis light absorbance. Spectroscopic detection methods include, but are not limited to, mass spectrometry, nuclear magnetic resonance (NMR) spectroscopy, and infrared spectroscopy. Electrostatic detection methods include, but are not limited to, gel based techniques, such as, for example, gel electrophoresis. Electrochemical detection methods include, but are not limited to, electrochemical detection of amplified product after high-performance liquid chromatography separation of the amplified products.

The terms “signal,” “signal sequence,” “sequence signal,” and “sequencing signal,” as used herein, generally refer to a series of signals (e.g., fluorescence measurements) associated with a DNA molecule or clonal population of DNA, comprising primary data. Such signals may be obtained using a high-throughput sequencing technology (e.g., flow sequencing-by-synthesis (SBS)). Such signals may be processed to obtain imputed sequences (e.g., during primary analysis).

The terms “sequence” or “sequence read,” as used herein, generally refer to a series of nucleotide assignments (e.g, by base calling) made during a sequencing process. Such sequences may be derived from signal sequences (e.g., during primary analysis). Sequence reads may be estimated or imputed sequence reads made by making preliminary base calls based on signal sequences, and the estimated or imputed sequence reads may then be subject to further base calling analysis or correction to produce final sequence reads (e.g., using the signal-to-noise (SNR) enhancement techniques disclosed herein).

The term “homopolymer,” as used herein, generally refers to a sequence of 0, 1, 2, . . . , N sequential nucleotides. For example, a homopolymer containing sequential A nucleotides may be represented as A, AA, AAA, . . . , up to N sequential A nucleotides.

The term “HpN truncation,” as used herein, generally refers to a method of processing a set of one or more sequences such that each homopolymer of the set of one or more sequences having a length greater than or equal to an integer N is truncated to a homopolymer of length N. For example, HpN truncation of the sequence “AGGGGGT” to 3 bases may result in a truncated sequence of “AGGGT.”

The term “analog alignment,” as used herein, generally refers to alignment of signal sequences to a reference signal sequence.

The term “context dependence” or “context dependency,” as used herein, generally refers to signal correlations with local sequence, relative nucleotide representation, or genomic locus. Signals for a given sequence may vary due to context dependency, which may depend on the local sequence, relative nucleotide representation of the sequence, or genomic locus of the sequence.

The goal to elucidate the entire human genome has created interest in technologies for rapid nucleic acid (e.g., DNA) sequencing, both for small and large scale applications. As knowledge of the genetic basis for human diseases increases, high-throughput DNA sequencing has been leveraged for myriad clinical applications. Despite the prevalence of nucleic acid sequencing methods and systems in a wide range of molecular biology and diagnostics applications, such methods and systems may encounter challenges in accurate base calling. In particular, sequencing methods that perform base calling based on quantified characteristic signals indicating nucleotide incorporation can have sequencing errors, for example, stemming from fundamental random errors (e.g., Poisson noise in detection and binomial noise from biochemistry processes) and/or unpredictable systematic variations in signal levels and context dependent signals that may be different for every sequence. Such signal variations and context dependency signals may cause issues with sequence calling.

Recognized herein is a need for improved base calling of sequences that addresses at least the abovementioned problems. Methods and systems provided herein can significantly reduce or eliminate errors in base calling and/or homopolymer length assessment of sequences resulting from fundamental random errors (e.g., Poisson noise in detection and binomial noise from biochemistry processes), which can generally be reduced by the square root of the number of replicates. Methods and systems of the present disclosure may use molecular barcodes to group sequencing signals, aggregate sequencing signals within groups, and combine aggregated sequencing signals to generate consensus sequences. Such methods and systems may achieve accurate and efficient base calling of sequences and/or homopolymer length assessment with very low single-copy error rates, which are required to maximize sensitivity of detecting rare events (e.g., rare instance of a sequence or partial sequence) while maximizing specificity (e.g., minimizing false detections).

Flow sequencing by synthesis (SBS) procedures typically comprise performing repeated DNA extension cycles, wherein individual species of nucleotides and/or labeled analogs are sequentially presented to a primer-template-polymerase complex, which then incorporates the nucleotide if complementary (to a growing strand in the primer-template-polymerase complex). The product of each flow may be measured for each clonal population of templates, e.g., a bead or a colony. The resulting nucleotide incorporations may be detected and quantified by unambiguously distinguishing signals corresponding to or associated with zero, one, or more sequential incorporations. Where the same species of nucleotide (e.g., of a canonical base type) is complementary to consecutive positions on the growing strand (e.g., in a homopolymer segment), a flow may result in multiple incorporations into the growing strand. Accurate base calling and/or homopolymer length assessment of sequences may comprise quantification of such multiple sequential incorporations, which may comprise quantifying characteristic signals for each possible case of 0, 1, 2, . . . , N sequential nucleotides incorporated on a colony in each flow. For example, a set of sequential A nucleotides may be represented as A, AA, AAA, . . . , up to N sequential A nucleotides.

In some cases, accurate base calling and/or homopolymer length assessment of sequences may encounter challenges owing to fundamental random errors (e.g., Poisson noise in detection and binomial noise from biochemistry processes, which can generally be reduced by the square root of the number of replicates) and/or unpredictable systematic variations in signal level, any of which can cause errors in base calling. In some cases, instrument and detection systematics can be calibrated and removed by monitoring instrument diagnostics and common-mode behavior across large numbers of colonies. Accurate base calling and/or homopolymer length assessment of sequences may also encounter challenges owing to sequence context dependent signal, which may be different for every sequence. For example, in the case of fluorescence measurements of dilute labeled nucleotides, sequence context can affect both the number of labeled analogs (variable tolerance for incorporating labeled analogs) as well as fluorescence of individual labeled analogs (e.g., quantum yield of dyes affected by local context of ±5 bases, as described by [Kretschy, et al., Sequence-Dependent Fluorescence of Cy3- and Cy5-Labeled Double-Stranded DNA, Bioconjugate Chem., 27(3), pp. 840-848], which is incorporated herein by reference in its entirety). In practice, with dye-terminator Sanger cycle sequencing, substantial systematic variations in signals have been identified for 3-base contexts (e.g., as described by [Zakeri, et al., Peak height pattern in dichloro-rhodamine and energy transfer dye terminator sequencing, Biotechniques, 25(3), pp. 406-10], which is incorporated herein by reference in its entirety).

The present disclosure provides methods and systems for improved base calling and/or homopolymer length assessment of sequences using molecular barcodes for efficient analog signal enhancement via barcode grouping toward sequencing applications (e.g., suitable for flow SBS). The methods and systems may comprise algorithmic steps to accurately and efficiently determine base calls and/or homopolymer lengths from a given series of sequence signals corresponding to nucleotide flows.

In various aspects, such as cases where individual sequence signals have poor signal-to-noise ratio (SNR) that may cause poor base accuracy contributing to inaccurate genomic alignment, methods and systems of the present disclosure can be applied to boost SNR of such sequence signals prior to final base-calling. These methods and systems may comprise obtaining a sample of input nucleic acid molecules, attaching barcodes from among a plurality of different barcodes to individual input nucleic acid molecules to produce a plurality of barcoded nucleic acid molecules, and amplifying the plurality of barcoded nucleic acid molecules to produce a library of amplicons. This library may comprise exact copy fragments (having the same barcode and sequence) of the initial plurality of barcoded nucleic acid molecules, as well as allele copies and allele variants thereof, which may generally share molecular barcodes and fragment endpoints (e.g., starting points and ending points). Methods and systems of the present disclosure may comprise grouping exact copy fragments together (e.g., which have been amplified from the same initial template molecule), and aggregating or combining their signals within a group to significantly enhance the SNR of sequence signals, thereby enabling more accurate base calling and/or homopolymer length assessment.

One approach to performing such SNR enhancement of sequence signals may comprise comparing all of the plurality of N sequence reads with each other, and grouping the best matches together. However, such an approach can be computationally expensive, since the computational complexity of this operation may be of order N² (in big-O notation), which may be computationally problematic when N is very large (e.g., on the order of 1 billion input nucleic acid sample fragments, which is a nominal amount for applications such as human whole genome sequencing).

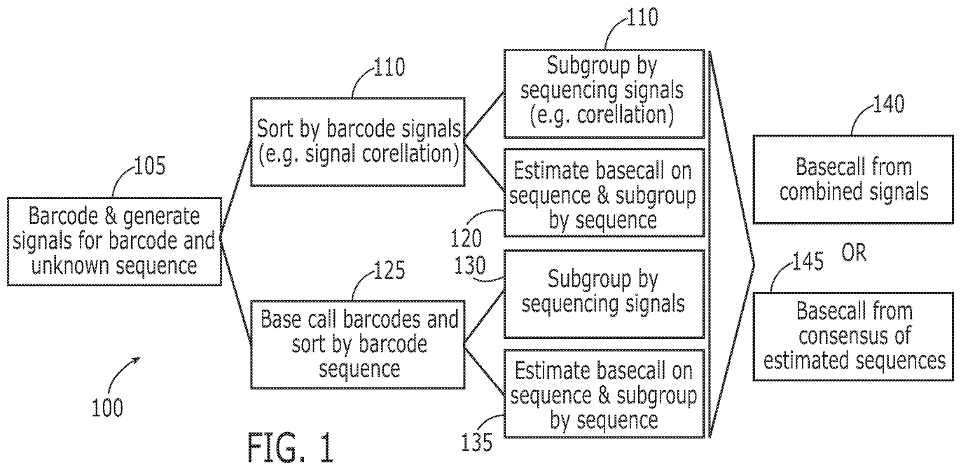

FIG. 1 shows an example of a flowchart illustrating a method 100 of base calling using molecular barcodes, in accordance with disclosed embodiments. First, a plurality of initial template molecules may be barcoded, and signals of the barcodes and unknown sequences of the initial template molecules may be generated (as in 105). Next, the unknown sequences of the initial template molecules may be sorted by barcoded signals (e.g., by signal correlation) (as in 110), and then further subgrouped by sequencing signals (e.g., by correlation) (as in 115) or based on estimated base calls of the unknown sequence (as in 120). Alternatively, the unknown sequences of the initial template molecules may be sorted based on barcode sequences (e.g., generated by base calls of the barcode signals) (as in 125), and then further subgrouped by sequencing signals (as in 130) or based on estimated base calls of the unknown sequence (as in 135). Finally base calls of the unknown sequence can be made from the combined signals (as in 140) or from base calls from a consensus of the estimated sequences (as in 145).

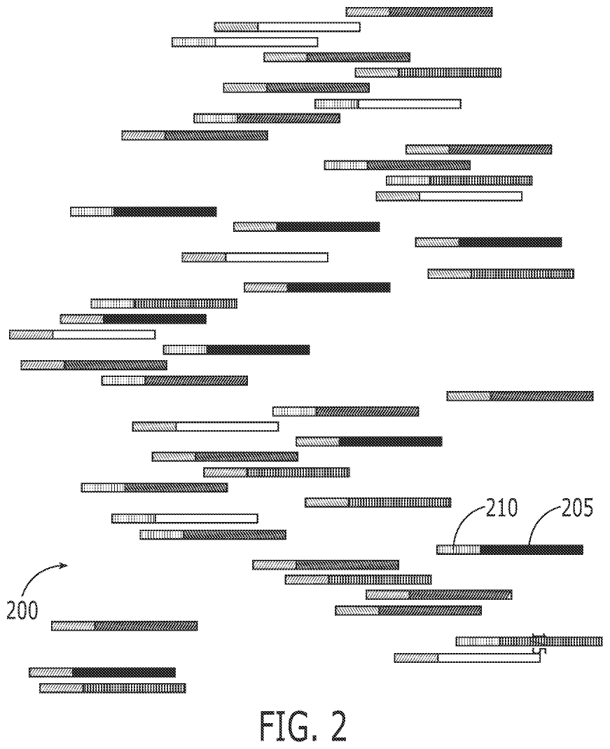

As shown in FIG. 2, methods and systems of the present disclosure may comprise preparing the input sample of nucleic acid molecules 200 whereby each initial template molecule of the input sample of nucleic acid molecules 205 is ligated to one of a plurality of barcodes 210. In some embodiments, each initial template molecule 205 of the input sample of nucleic acid molecules 200 is uniquely ligated to one of a plurality of barcodes 210, thereby producing a plurality of barcoded nucleic acid molecules each having different barcodes (e.g., such that any pair of the plurality of barcoded nucleic acid molecules is attached or ligated to different barcodes).

After barcoding the plurality of initial template molecules, the plurality of barcoded nucleic acid molecules may be amplified to a sufficient extent (e.g., number of amplification cycles) such that there is a reasonable likelihood (e.g., at least about 50%, at least about 60%, at least about 70%, at least about 80%, at least about 90%, at least about 95%, at least about 96%, at least about 97%, at least about 98%, at least about 99%, at least about 99.9%, or at least about 99.99%) of obtaining a mean number of more than one exact copy (e.g., number of amplicons) for each initial template molecule.

Methods of the present disclosure may be performed without aligning imputed sequence reads among the entire plurality of imputed sequence reads to each other (e.g., against each other imputed sequence read among the entire plurality of imputed sequence reads), thereby reducing the computational complexity of the base calling and/or homopolymer length assessment. Alternatively, methods of the present disclosure may be performed without aligning sequence signals among the entire plurality of sequence signals to each other (e.g., against each other sequence signal among the entire plurality of sequence signals), thereby reducing the computational complexity of the base calling and/or homopolymer length assessment.

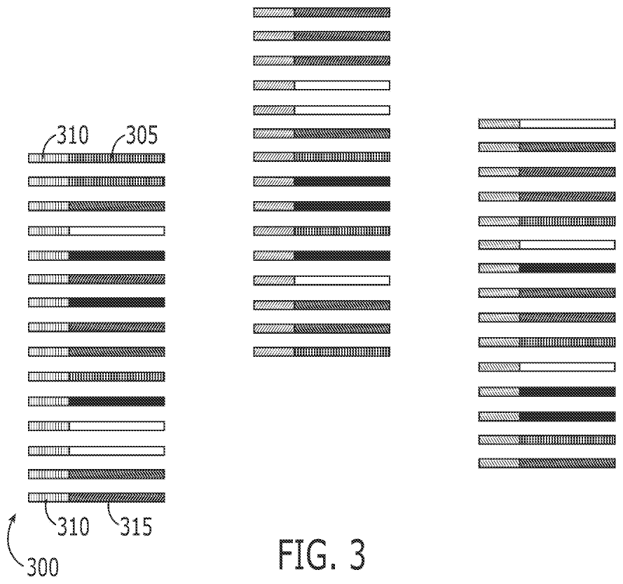

In some embodiments, each sequence signal or imputed sequence read may be classified or grouped according to its barcode signal (e.g., analog signal or imputed sequence read corresponding to a molecular barcode attached to the fragment from which the imputed sequence read was generated) into different barcode pools (e.g., a barcode pool 300), as shown in FIG. 3 (with each fragment containing a longer input sequence corresponding to the initial template molecule 305, and a shorter barcode sequence corresponding to the ligated molecular barcode 310). Since a barcode pool 300 may comprise sequence signals or imputed sequence reads having the same molecular barcode 310, the sequence signals or imputed sequence reads may be interpreted or treated in subsequent analyses as possibly arising from the same initial template molecule of the input sample of nucleic acid molecules. The sequence signals or imputed sequence reads within a barcode pool 300 may also correspond to different initial template molecules (e.g., having sequences 305 and 315) of the input sample of nucleic acid molecules. The grouping can be performed based on an analog classification (e.g., grouping together sequence signals having analog signals with the same molecular barcode) or based on digitizing the barcode (e.g., grouping together imputed sequence reads having the same molecular barcode).

In some embodiments, the plurality of barcodes can comprise a sufficient number of bases given the molecular diversity of the input sample, such that the initial template molecules can be uniquely or non-uniquely tagged and identified. The plurality of barcodes can comprise 1 base, 2 bases, 3 bases, 4 bases, 5 bases, 6 bases, 7 bases, 8 bases, 9 bases, 10 bases, 11 bases, 12 bases, 13 bases, 14 bases, 15 bases, 16 bases, 17 bases, 18 bases, 19 bases, 20 bases, or more than 20 bases. Generally, a plurality of N-base barcodes may be sufficient to uniquely barcode a sample having about 4^{N} initial template molecules.

In some embodiments, the plurality of barcodes can be designed such that edit distances (e.g., Hamming distances) between any pair of barcodes among the plurality of barcodes are sufficient to avoid confusion (e.g., arising from single-base or few-base errors in amplification, replication, sequencing, base calling, and/or homopolymer length assessment), thereby enabling error detection and/or error correction of errors comprising 1 base, 2 bases, 3 bases, 4 bases, 5 bases, 6 bases, 7 bases, 8 bases, 9 bases, 10 bases, 11 bases, 12 bases, 13 bases, 14 bases, 15 bases, 16 bases, 17 bases, 18 bases, 19 bases, 20 bases, or more than 20 bases. In some embodiments, the plurality of barcodes can be designed such that a subset of the number of bases of the barcodes is used for error checking or correction (ECC) purposes (e.g., similar to the use of parity bits in data communications).

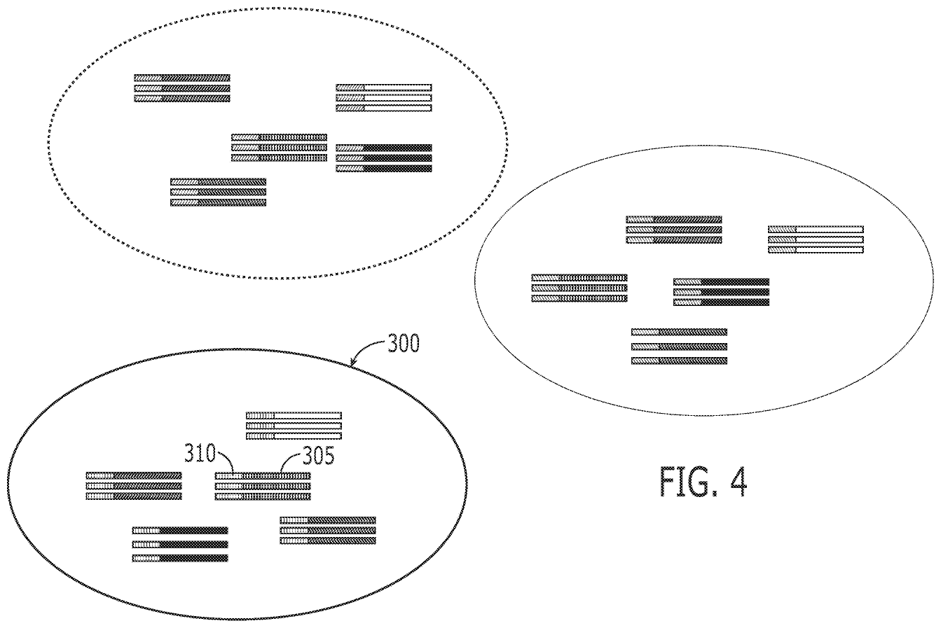

As shown in FIG. 4, after the sequence signals or imputed sequence reads of the barcoded library fragments are grouped into barcode groups (e.g., barcode pool 300), the sequence signals or imputed sequence reads within each barcode group may be compared to each other (e.g., correlated), and identical sequence signals or imputed sequence reads may be identified and further grouped (e.g., within a barcode group) into families that are representative of the same initial template molecule (e.g., a family of three identical sequence signals or imputed sequence reads 305 having the same barcode 310). After this grouping into families by initial template molecule, the aligned sequence signals or imputed sequence reads can be combined within each family to produce a single sequence signal with higher SNR (e.g. average) for each family. This combined sequence signal or imputed sequence read can be base-called, aligned more accurately, and assessed for genetic variants with greater confidence than individual sequence signals or imputed sequence reads having lower SNR. Because these individual sequence signals or imputed sequence reads have originated from a single initial template molecule, they represent a single allele, substantially simplifying analysis. In some embodiments, this process can be accomplished with only analog signal processing steps up to base calling.

As a numeric example of the computation efficiency, suppose a plurality of 10⁹ individual imputed sequence reads that are barcoded with a plurality of 10⁵ barcodes are processed. Performing a naïve read-to-read alignment may require an order of O(10¹⁸) correlation operations. In comparison, methods of the present disclosure may be performed to process the same plurality of 10⁹ individual imputed sequence reads that are barcoded with a plurality of 10⁵ barcodes, by performing 10⁹ barcode classification operations, followed by 10⁵(10⁹/10⁵)²=10¹³ correlation operations; thereby achieving a reduction in computation by a factor equal to the diversity of the barcode library (e.g., in this case, 5 orders of magnitude or a factor of 10,000). Therefore, methods of the present disclosure can be used advantageously to perform rare variant calls based on few or single input copies of initial template nucleic acid molecules, thereby achieving significant gains in efficiency as well as accuracy of base calling and/or homopolymer length assessment due to the analog signal enhancement approach.

### Efficient Analog Signal Enhancement Using Repeated SBS on Colonies

In some embodiments, methods of the present disclosure may comprise reducing random signal variation arising from chemistry and detection processes, by performing sequencing-by-synthesis (SBS) (or similar) sequencing of clusters, followed by denaturation of the synthesized copies and a second sequencing process. The random variations in detection and chemistry associated with the second SBS operation may be independent and can be averaged with the first signals to reduce noise. This process can be repeated as necessary to reduce random error to a desired or target level. An advantage of this approach may include incurring only the preparation and substrate costs for a single copy, although the scanning and SBS costs are multiplied as with the parallel copy method described above.

In various aspects of the present disclosure, methods for sequencing a plurality of nucleic acid molecules may comprise (i) sorting by sequence signals or barcode sequence, (ii) subgrouping by sequence signals or barcode sequences, and aggregating the sequence signals or barcode sequences within subgroups. The method for sequencing a plurality of nucleic acid molecules may comprise using a plurality of barcode molecules to barcode a plurality of nucleic acid molecules from a biological sample, to generate a plurality of barcoded nucleic acid molecules comprising a plurality of barcode sequences. Next, the method may comprise sequencing the plurality of barcoded nucleic acid molecules to generate a plurality of sequencing signals. The plurality of sequencing signals may comprise signals corresponding to the plurality of barcode sequences, and the plurality of sequencing signals may not be sequencing reads. Alternatively, the method may comprise sequencing the plurality of barcoded nucleic acid molecules to generate a plurality of imputed sequence reads.

Next, the method may comprise using the signals corresponding to the plurality of barcode sequences to group the plurality of sequencing signals into a plurality of groups. The sequencing signals of a given group of the plurality of groups may comprise signals corresponding to a barcode sequence of the plurality of barcode sequences that is (i) identical for the given group and (ii) different from barcode sequences of other groups of the plurality of groups. Alternatively, the method may comprise using the imputed sequence reads corresponding to the plurality of barcode sequences to group the plurality of imputed sequence reads into a plurality of groups. The imputed sequence reads of a given group of the plurality of groups may comprise a barcode sequence of the plurality of barcode sequences that is (i) identical for the given group and (ii) different from barcode sequences of other groups of the plurality of groups.

Next, the method may comprise processing the sequencing signals within the given group to generate one or more sets of aggregated signals. The one or more sets of aggregated signals may not be sequencing reads. Next, the method may comprise combining the one or more sets of aggregated signals to generate a consensus sequence for the nucleic acid molecule. Alternatively, the method may comprise aggregating the imputed sequence reads within the given group to generate one or more sets of aggregated sequence reads.

### Base Calling Via Sorting by Barcode Signals and Subgrouping by Sequencing Signals

In an aspect, the present disclosure provides a method for sequencing a plurality of nucleic acid molecules, comprising: (a) using a plurality of barcode molecules to barcode a plurality of nucleic acid molecules from a biological sample, to generate a plurality of barcoded nucleic acid molecules comprising a plurality of barcode sequences; (b) sequencing the plurality of barcoded nucleic acid molecules to generate a plurality of sequencing signals, which plurality of sequencing signals comprises signals corresponding to the plurality of barcode sequences, wherein the plurality of sequencing signals are not sequencing reads; (c) using the signals corresponding to the plurality of barcode sequences to group the plurality of sequencing signals into a plurality of groups, wherein sequencing signals of a given group of the plurality of groups comprise signals corresponding to a barcode sequence of the plurality of barcode sequences that is (i) identical for the given group and (ii) different from barcode sequences of other groups of the plurality of groups; (d) processing the sequencing signals within the given group to generate one or more sets of aggregated signals, wherein the one or more sets of aggregated signals are not sequencing reads; and (e) combining the one or more sets of aggregated signals to generate a consensus sequence.

In some embodiments, the combining in (e) comprises performing base calling to identify individual bases. The base calling may be performed by processing aggregated signals within each of the one or more sets of aggregated signals to each other to generate the consensus sequence. In some embodiments, the method further comprises averaging the aggregated signals within each of the one or more sets of aggregated signals to each other to generate the consensus sequence. The consensus sequence may be compared to a reference to identify one or more genetic variants.

In some embodiments, the plurality of nucleic acid molecules, which may include DNA (e.g., methylated DNA) molecules or RNA molecules, is obtained from a bodily sample of a subject. The barcoding may comprise ligating the barcode molecules to the plurality of nucleic acid molecules. The plurality of barcoded nucleic acid molecules may be uniquely or non-uniquely barcoded. In some embodiments, the plurality of barcode molecules comprises at least about 10, at least about 100, at least about 1,000, at least about 10,000, or at least about 100,000 distinct barcodes. In some embodiments, the plurality of sequencing signals comprises analog signals. In some embodiments, the method further comprises, pre-processing the plurality of sequencing signals to remove systematic errors. In some embodiments, the method further comprises, prior to (b), amplifying the plurality of barcoded nucleic acid molecules (e.g., by PCR or RPA). In some embodiments, steps (c), (d), and/or (e) are performed in real time or near real time with the sequencing of (b).

In another aspect, the present disclosure provides a system for sequencing a plurality of nucleic acid molecules, comprising: a database that stores a plurality of sequencing signals generated upon using a plurality of barcode molecules to barcode the plurality of nucleic acid molecules and sequencing the plurality of barcoded nucleic acid molecules, which plurality of sequencing signals comprises signals corresponding to the plurality of barcode sequences, wherein the plurality of sequencing signals are not sequencing reads; and one or more computer processors operatively coupled to the database, wherein the one or more computer processors are individually or collectively programmed to: use the signals corresponding to the plurality of barcode sequences to group the plurality of sequencing signals into a plurality of groups, wherein sequencing signals of a given group of the plurality of groups comprise signals corresponding to a barcode sequence of the plurality of barcode sequences that is (i) identical for the given group and (ii) different from barcode sequences of other groups of the plurality of groups; process the sequencing signals within the given group to generate one or more sets of aggregated signals, wherein the one or more sets of aggregated signals are not sequencing reads; and combine the one or more sets of aggregated signals to generate a consensus sequence.

In some embodiments, a plurality of imputed sequences and their associated sequence signals may be aggregated to identify a local context. The plurality of imputed sequences and their associated sequence signals may then be stacked together, in some cases using alignment to a reference genome, in order to identify and group nucleotide bases associated with the same genomic positions. The plurality of imputed sequences and their associated sequence signals may be stacked together by comparison of the imputed sequences to each other to identify common local contexts. Alternatively, the plurality of imputed sequences and their associated sequence signals may be stacked together by alignment to a reference sequence. For example, the plurality of imputed sequences (and their associated sequence signals) may be aligned to a reference genome (e.g., a human reference genome, such as hg19 or hg38). Alternatively, the plurality of sequence signals (and their associated imputed sequences) may be aligned to a reference signal. The stacked imputed sequences and their associated signals may be stacked together using any number of consecutive bases that are likely to contain context dependency, such as 2 bases, 3 bases, 4 bases, 5 bases, 6 bases, 7 bases, 8 bases, 9 bases, 10 bases, 11 bases, 12 bases, 13 bases, 14 bases, 15 bases, 16 bases, 17 bases, 18 bases, 19 bases, 20 bases, or more than 20 bases.

Using these imputed sequences, which may be aggregated and grouped according to their molecular barcodes and/or an n-base local context (e.g., a number of n consecutive bases located proximate to the imputed sequence), a context model can be built and trained (e.g., by aggregating data for a particular genomic context to observe any systematic behavior) to learn how to interpret signals toward accurate base calling. Developing a context model may comprise analyzing the plurality of associated sequence signals to discover systematic behavior, and developing rules for predicting base calls, based on correlations between context-dependent signals and imputed sequences, as described elsewhere herein. Such correlations, or context dependencies, may comprise a number of bases (e.g., 2 bases, 3 bases, 4 bases, 5 bases, 6 bases, 7 bases, 8 bases, 9 bases, 10 bases, 11 bases, 12 bases, 13 bases, 14 bases, 15 bases, 16 bases, 17 bases, 18 bases, 19 bases, 20 bases, or more than 20 bases) prior to and/or after a given sequence or signal. For example, if an ‘A’ appears after a first sequence (e.g., ‘TCTCG’), based on context dependency, a first signal level (e.g., 0.7 of the nominal signal) may be expected, and if the ‘A’ appears after a second sequence (e.g., ‘AAACC’), a second signal level (e.g., 1.3 of the nominal signal may be expected). Such context dependency can be aggregated into a trained model to refine, for example, base calls from imputed sequences and/or sequence signals.

For example, the context model may be built and trained (e.g., using machine learning techniques) based on analysis of imputed sequences and associated signals obtained by sequencing DNA molecules with known sequences (e.g., from synthetic template DNA molecules). Such a context model may comprise expected sequence signals (e.g., signal amplitudes) corresponding to an n-base portion of a locus (e.g., where N is at least 1 base, at least 2 bases, at least 3 bases, at least 4 bases, at least 5 bases, at least 6 bases, at least 7 bases, at least 8 bases, at least 9 bases, or at least 10 bases). Alternatively, or in addition, context models may comprise or incorporate distributions, medians, averages, modes, standard deviations, quantiles, interquartile ranges, or other quantitative or statistical measures of sequence signals (e.g., signal amplitudes) corresponding to an n-base portion of a locus.

Methods and systems of the present disclosure may comprise algorithms that use only a sequence known a priori (e.g., a double-stranded sequence), or simultaneously assessing a series of flow measurements to determine a series of base calls comprising a sequence most likely to produce the observations (e.g., a maximum likelihood sequence determination). The algorithms may account for any label-label interactions, e.g. quenching, that may occur and influence the sequence signals. The algorithms may also account for any known position-dependent signal and/or any photobleaching effects that may occur and influence the sequence signals. For example, context dependency may be affected by flow sequencing of mixed populations of nucleotides (e.g., comprising natural nucleotides and modified nucleotides). Such mixed populations of nucleotides may compete for incorporation by a polymerase in a flow sequencing process, thereby giving rise to varying context-dependent sequence signals.

The algorithms may incorporate training data of known sequences comprising at one or more replicates of every context having significant correlation with homopolymer signal variation. Such incorporation may be repeated for every different discrete chemistry variant for which the algorithm is to be applied.

The algorithms may comprise auxiliary outputs, which may include assessments of the quantization noise (e.g., Poisson or binomial random variation) or other quality assessments, including a confidence interval or error assessment of the homopolymer length. The outputs may also include dynamic assessments of chemistry process parameters (e.g., temperature) and the most likely labeling fraction to account for the observations as well.

The trained context model may then be applied by one or more trained algorithms (e.g., machine learning algorithms) to predict base calls (such as, for example, of a plurality of imputed sequences and associated signals obtained by sequencing DNA molecules with unknown sequences). Such predictions may comprise refining or correcting base calls of a plurality of imputed sequences. Alternatively, such predictions may comprise determining base calls from a plurality of sequence signals. For example, a second set of DNA molecules comprising unknown sequences may be sequenced, thereby generating a second plurality of sequence signals and imputed sequences. Next, base calls of the second set of DNA molecules may be generated, e.g., based at least on (i) the second plurality of imputed sequences and/or sequence signals associated with the second plurality of sequence signals, (ii) the second plurality of imputed sequences, (iii) at least a portion of the expected signals, (iv) the known sequence, or (v) a combination thereof. In some embodiments, such predictions may be performed in real-time (e.g., as sequence signals are measured). For example, real-time can include a response time of less than 1 second, tenths of a second, hundredths of a second, a millisecond, or less. Real-time can include a simultaneous or substantially simultaneous process or operation (e.g., generating base calls) happening relative to another process or operation (e.g., measuring sequence signals). All of the operations described herein, such as training an algorithm, predicting and/or generating base calls and other operations, such as those described elsewhere herein, can be configured to be capable of happening or being performed in real-time.

### Base Calling Via Sorting by Barcode Sequences and Subgrouping by Sequencing Signals

In another aspect, the present disclosure provides a method for sequencing a plurality of nucleic acid molecules, comprising: (a) using a plurality of barcode molecules to barcode a plurality of nucleic acid molecules from a biological sample, to generate a plurality of barcoded nucleic acid molecules comprising a plurality of barcode sequences; (b) sequencing the plurality of barcoded nucleic acid molecules to generate a plurality of sequencing signals, which plurality of sequencing signals comprises signals corresponding to the plurality of barcode sequences, wherein the plurality of sequencing signals are not sequencing reads; (c) processing the signals corresponding to the plurality of barcode sequences to identify the barcode sequences of each of the plurality of sequencing signals; (d) using the identified barcode sequences to group the plurality of sequencing signals into a plurality of groups, wherein sequencing signals of a given group of the plurality of groups correspond to an identified barcode sequence of the plurality of barcode sequences that is (i) identical for the given group and (ii) different from identified barcode sequences of other groups of the plurality of groups; (e) processing the sequencing signals within the given group to generate one or more sets of aggregated signals, wherein the one or more sets of aggregated signals are not sequencing reads; and (f) combining the one or more sets of aggregated signals to generate a consensus sequence.

In some embodiments, in (f), the combining comprises performing base calling to identify individual bases. The base calling may be performed by processing aggregated signals within each of the one or more sets of aggregated signals to each other to generate the consensus sequence. In some embodiments, the method further comprises averaging the aggregated signals within each of the one or more sets of aggregated signals to each other to generate the consensus sequence. The consensus sequence may be compared to a reference to identify one or more genetic variants.

In some embodiments, the plurality of nucleic acid molecules, which may include DNA (e.g., methylated DNA) molecules or RNA molecules, is obtained from a bodily sample of a subject. The barcoding may comprise ligating the barcode molecules to the plurality of nucleic acid molecules. The plurality of barcoded nucleic acid molecules may be uniquely or non-uniquely barcoded. In some embodiments, the plurality of barcode molecules comprises at least about 10, at least about 100, at least about 1,000, at least about 10,000, or at least about 100,000 distinct barcodes. In some embodiments, the plurality of sequencing signals comprises analog signals. In some embodiments, the method further comprises, pre-processing the plurality of sequencing signals to remove systematic errors. In some embodiments, the method further comprises pre-processing the plurality of sequencing signals to remove systematic errors. In some embodiments, the method further comprises, prior to (b), amplifying the plurality of barcoded nucleic acid molecules (e.g., by PCR or RPA). In some embodiments, steps (d), (e), and/or (f) are performed in real time or near real time with the sequencing of (b).

In another aspect, the present disclosure provides a system for sequencing a plurality of nucleic acid molecules, comprising: a database that stores a plurality of sequencing signals generated upon using a plurality of barcode molecules to barcode the plurality of nucleic acid molecules and sequencing the plurality of barcoded nucleic acid molecules, which plurality of sequencing signals comprises signals corresponding to the plurality of barcode sequences, wherein the plurality of sequencing signals are not sequencing reads; and one or more computer processors operatively coupled to the database, wherein the one or more computer processors are individually or collectively programmed to: process the signals corresponding to the plurality of barcode sequences to identify the barcode sequences of each of the plurality of sequencing signals; use the identified barcode sequences to group the plurality of sequencing signals into a plurality of groups, wherein sequencing signals of a given group of the plurality of groups correspond to an identified barcode sequence of the plurality of barcode sequences that is (i) identical for the given group and (ii) different from identified barcode sequences of other groups of the plurality of groups; process the sequencing signals within the given group to generate one or more sets of aggregated signals, wherein the one or more sets of aggregated signals are not sequencing reads; and combine the one or more sets of aggregated signals to generate a consensus sequence.

### Base Calling Via Sorting by Barcode Signals and Subgrouping by Sequences

In another aspect, the present disclosure provides a method for sequencing a plurality of nucleic acid molecules, comprising: (a) using a plurality of barcode molecules to barcode a plurality of nucleic acid molecules from a biological sample, to generate a plurality of barcoded nucleic acid molecules comprising a plurality of barcode sequences; (b) sequencing the plurality of barcoded nucleic acid molecules to generate a plurality of sequencing signals, which plurality of sequencing signals comprises signals corresponding to the plurality of barcode sequences, wherein the plurality of sequencing signals are not sequencing reads; (c) using the signals corresponding to the plurality of barcode sequences to group the plurality of sequencing signals into a plurality of groups, wherein sequencing signals of a given group of the plurality of groups comprise signals corresponding to a barcode sequence of the plurality of barcode sequences that is (i) identical for the given group and (ii) different from barcode sequences of other groups of the plurality of groups; (d) processing the sequencing signals within the given group to generate one or more estimated sequences, wherein each of the one or more estimated sequences comprises a plurality of estimated base calls; and (e) combining the one or more estimated sequences to generate a consensus sequence.

In some embodiments, the one or more estimated sequences comprise a plurality of estimated sequences, and the consensus sequence is generated based on a majority vote among the plurality of estimated sequences. The consensus sequence may be compared to a reference to identify one or more genetic variants. In some embodiments, the plurality of nucleic acid molecules, which may include DNA (e.g., methylated DNA) molecules or RNA molecules, is obtained from a bodily sample of a subject. The barcoding may comprise ligating the barcode molecules to the plurality of nucleic acid molecules. The plurality of barcoded nucleic acid molecules may be uniquely or non-uniquely barcoded. In some embodiments, the plurality of barcode molecules comprises at least about 10, at least about 100, at least about 1,000, at least about 10,000, or at least about 100,000 distinct barcodes. In some embodiments, the plurality of sequencing signals comprises analog signals. In some embodiments, the method further comprises pre-processing the plurality of sequencing signals to remove systematic errors. In some embodiments, the method further comprises, prior to (b), amplifying the plurality of barcoded nucleic acid molecules (e.g., by PCR or RPA). In some embodiments, steps (c), (d), and/or (e) are performed in real time or near real time with the sequencing of (b).

In another aspect, the present disclosure provides a system for sequencing a plurality of nucleic acid molecules, comprising: a database that stores a plurality of sequencing signals generated upon using a plurality of barcode molecules to barcode the plurality of nucleic acid molecules and sequencing the plurality of barcoded nucleic acid molecules, which plurality of sequencing signals comprises signals corresponding to the plurality of barcode sequences, wherein the plurality of sequencing signals are not sequencing reads; and one or more computer processors operatively coupled to the database, wherein the one or more computer processors are individually or collectively programmed to: use the signals corresponding to the plurality of barcode sequences to group the plurality of sequencing signals into a plurality of groups, wherein sequencing signals of a given group of the plurality of groups comprise signals corresponding to a barcode sequence of the plurality of barcode sequences that is (i) identical for the given group and (ii) different from barcode sequences of other groups of the plurality of groups; process the sequencing signals within the given group to generate one or more estimated sequences, wherein each of the one or more estimated sequences comprises a plurality of estimated base calls; and combine the one or more estimated sequences to generate a consensus sequence.

### Base Calling Via Sorting by Barcode Sequences and Subgrouping by Sequences

In another aspect, the present disclosure provides a method for sequencing a plurality of nucleic acid molecules, comprising: (a) using a plurality of barcode molecules to barcode a plurality of nucleic acid molecules from a biological sample, to generate a plurality of barcoded nucleic acid molecules comprising a plurality of barcode sequences; (b) sequencing the plurality of barcoded nucleic acid molecules to generate a plurality of sequencing signals, which plurality of sequencing signals comprises signals corresponding to the plurality of barcode sequences, wherein the plurality of sequencing signals are not sequencing reads; (c) processing the signals corresponding to the plurality of barcode sequences to identify the barcode sequences of each of the plurality of sequencing signals; (d) using the identified barcode sequences to group the plurality of sequencing signals into a plurality of groups, wherein sequencing signals of a given group of the plurality of groups correspond to an identified barcode sequence of the plurality of barcode sequences that is (i) identical for the given group and (ii) different from barcode sequences of other groups of the plurality of groups; (e) processing the sequencing signals within the given group to generate one or more estimated sequences, wherein each of the one or more estimated sequences comprises a plurality of estimated base calls; and (f) combining the one or more estimated sequences to generate a consensus sequence.

In some embodiments, the one or more estimated sequences comprise a plurality of estimated sequences, and the consensus sequence is generated based on a majority vote among the plurality of estimated sequences. In some embodiments, the method further comprises processing the consensus sequence against a reference to identify one or more genetic variants. In some embodiments, the plurality of nucleic acid molecules, which may include DNA (e.g., methylated DNA) molecules or RNA molecules, is obtained from a bodily sample of a subject. The barcoding may comprise ligating the barcode molecules to the plurality of nucleic acid molecules. The plurality of barcoded nucleic acid molecules may be uniquely or non-uniquely barcoded. In some embodiments, the plurality of barcode molecules comprises at least about 10, at least about 100, at least about 1,000, at least about 10,000, or at least about 100,000 distinct barcodes. In some embodiments, the plurality of sequencing signals comprises analog signals. In some embodiments, the method further comprises pre-processing the plurality of sequencing signals to remove systematic errors. In some embodiments, the method further comprises pre-processing the plurality of sequencing signals to remove systematic errors. In some embodiments, the method further comprises, prior to (b), amplifying the plurality of barcoded nucleic acid molecules (e.g., by PCR or RPA). In some embodiments, steps (d), (e), and/or (f) are performed in real time or near real time with the sequencing of (b).

In another aspect, the present disclosure provides a system for sequencing a plurality of nucleic acid molecules, comprising: a database that stores a plurality of sequencing signals generated upon using a plurality of barcode molecules to barcode the plurality of nucleic acid molecules and sequencing the plurality of barcoded nucleic acid molecules, which plurality of sequencing signals comprises signals corresponding to the plurality of barcode sequences, wherein the plurality of sequencing signals are not sequencing reads; and one or more computer processors operatively coupled to the database, wherein the one or more computer processors are individually or collectively programmed to: process the signals corresponding to the plurality of barcode sequences to identify the barcode sequences of each of the plurality of sequencing signals; use the identified barcode sequences to group the plurality of sequencing signals into a plurality of groups, wherein sequencing signals of a given group of the plurality of groups correspond to an identified barcode sequence of the plurality of barcode sequences that is (i) identical for the given group and (ii) different from identified barcode sequences of other groups of the plurality of groups; process the sequencing signals within the given group to generate one or more estimated sequences, wherein each of the one or more estimated sequences comprises a plurality of estimated base calls; and combine the one or more estimated sequences to generate a consensus sequence.

### Methods for Homopolymer Calling

Methods and systems of the present disclosure may be used to perform accurate and efficient base calling of sequences comprising homopolymers. Such base calling may be performed as part of a sequencing process, such as performing next-generation sequencing (e.g., sequencing by synthesis or flow sequencing) of nucleic acid molecules (e.g., DNA molecules). Such nucleic acid molecules may be obtained from or derived from a sample from a subject. Such a subject may have a disease or be suspected of having a disease. Methods and systems described herein may be useful for significantly reducing or eliminating errors in quantifying homopolymer lengths and errors associated with context dependence. Such methods and systems may achieve accurate and efficient base calling of homopolymers, quantification of homopolymer lengths, and quantification of context dependency in sequence signals.

The methods and systems provided herein may be used to directly call homopolymer lengths with high accuracy for each read. In addition, the methods and systems provided herein may comprise alignment of provisionally quantified reads (e.g., imputed or estimated sequences) containing homopolymers of uncertain length to a reference. Such alignment may be performed using an algorithm that places low penalty on homopolymer length errors. Using the statistical power of multiple aligned reads, the assessment of homopolymer lengths and uncertainties (e.g., confidence interval or error assessment), the methods and systems provided herein may determine the homopolymer lengths based on a consensus of all reads (e.g., for homozygous loci) or cluster reads. Alternatively or in combination, the methods and systems provided herein may make consensus calls on clusters (e.g., for heterozygous loci).

Methods of the present disclosure may comprise processing a plurality of sequence signals. Such a method may be used to determine homopolymer lengths by consensus of aligned reads, such as by alignment to a HpN-truncated reference sequence. The method may comprise sequencing a nucleic acid sample to provide a plurality of sequence signals and imputed sequences. From such imputed sequences, homopolymer sequences (e.g., a sequence containing a homopolymer comprising multiple consecutive nucleotides of the same base) of at least N bases may be identified. These identified imputed homopolymer sequences may then be truncated to a homopolymer sequence of bases of length N, to yield one or more HpN truncated sequences. The length N may be any number of a plurality of bases, such as 2 bases, 3 bases, 4 bases, 5 bases, 6 bases, 7 bases, 8 bases, 9 bases, 10 bases, 11 bases, 12 bases, 13 bases, 14 bases, 15 bases, or more than 15 bases. As an example of truncated homopolymer alignment, all identified homopolymers of length N or greater in a given sequence may be truncated to a homopolymer of length N and then aligned to a reference.

After truncation, the one or more HpN truncated sequences may be aligned to one or more truncated references. Such truncated references may be HpN truncated and thereby comprise one or more homopolymer sequences truncated to length N. After alignment of the one or more HpN truncated sequences, a consensus sequence may be generated from the one or more HpN truncated sequences aligned to the one or more HpN truncated references. Such a consensus sequence may comprise a homopolymer sequence of the length N. The consensus sequence may be generated based on the aligned HpN truncated sequences, the sequence signals associated with the aligned HpN truncated sequences, or a combination thereof.

In some embodiments, processing a plurality of sequence signals may comprise calculating a length estimation error of the homopolymer sequence. The length estimation error may comprise a confidence interval for the length of the homopolymer sequence (homopolymer length). For example, the length estimation error for a homopolymer with an imputed length of 5 bases may comprise a confidence interval of [3, 7], or 5 bases ±2 bases. The length estimation error may be calculated based at least on a distribution of signals or imputed homopolymer lengths of the one or more HpN truncated sequences aligned to the HpN truncated references.

In some embodiments, processing a plurality of sequence signals may comprise pre-processing the plurality of sequence signals to remove systematic errors. Such pre-processing may be performed prior to truncating identified imputed homopolymer sequences and aligning the HpN truncated sequences to one or more truncated references. The pre-processing may be performed to address random and unpredictable systematic variations in signal level, which can cause errors in quantifying the homopolymer length. In some cases, instrument and detection systematic variation can be calibrated and removed by monitoring instrument diagnostics and common-mode behavior across large numbers of colonies.

In some embodiments, processing a plurality of sequence signals may comprise determining lengths of the homopolymer sequences. This determining may be performed by determining the number of sequential nucleotides appearing in the consensus sequences generated from the aligned HpN truncated sequences associated with the plurality of sequence signals. This determining may be performed based at least on clustering of the homopolymer sequences or sequence signals associated with the homopolymer sequences.

In some embodiments, the plurality of sequence signals is generated by sequencing nucleic acids of a subject. The HpN truncated references may comprise an HpN truncated reference genome of a species of the subject (e.g., an HpN truncated human reference genome). In some cases, a number of lengths computed or classified when generating the consensus sequence may be restricted, based at least on the ploidy of the species of the subject. The plurality of sequence signals and/or imputed sequences may be generated by any suitable sequencing approach, such as massively parallel array sequencing, flow sequencing, sequencing by synthesis, or dye sequencing.

Methods of the present disclosure may comprise quantifying context dependency of a plurality of sequence signals and imputed sequences. Such a method may be used to quantify homopolymer lengths by extensive training with an essay on a known genome. The method may comprise sequencing deoxyribonucleic acid (DNA) molecules to provide a plurality of sequence signals and imputed sequences. In some cases, the DNA molecules comprise a known sequence. From such imputed sequences, homopolymer sequences (e.g., a sequence containing a homopolymer comprising multiple consecutive nucleotides of the same base) of at least N bases may be identified. These identified imputed homopolymer sequences may then be truncated to a homopolymer sequence of bases of length N, to yield one or more HpN truncated sequences. The length N may be any number of a plurality of bases, such as 2 bases, 3 bases, 4 bases, 5 bases, 6 bases, 7 bases, 8 bases, 9 bases, 10 bases, 11 bases, 12 bases, 13 bases, 14 bases, 15 bases, or more than 15 bases. After truncation, the one or more HpN truncated sequences may be aligned to one or more truncated references. Such truncated references may be HpN truncated and thereby comprise one or more homopolymer sequences truncated to length N. After alignment of the one or more HpN truncated sequences, context dependency of the associated sequence signals may be quantified. Such quantification may be based at least on (i) the one or more HpN truncated sequences aligned to the one or more HpN truncated references and/or sequence signals associated with the one or more HpN truncated sequences aligned to the HpN truncated references, (ii) the known sequence, or (iii) a combination thereof.

In some embodiments, quantifying context dependency of a plurality of sequence signals and imputed sequences comprises sequencing a second set of DNA molecules comprising unknown sequences, thereby generating a second plurality of sequence signals and imputed sequences. From such imputed sequences, second homopolymer sequences (e.g., a sequence containing a homopolymer comprising multiple consecutive nucleotides of the same base) of at least N bases may be identified. These identified imputed second homopolymer sequences may then be truncated to a homopolymer sequence of bases of length N, to yield one or more second HpN truncated sequences. The length N may be any number of a plurality of bases, such as 2 bases, 3 bases, 4 bases, 5 bases, 6 bases, 7 bases, 8 bases, 9 bases, 10 bases, 11 bases, 12 bases, 13 bases, 14 bases, 15 bases, or more than 15 bases. After truncation, the one or more second HpN truncated sequences may be aligned to the one or more HpN truncated references. After alignment of the one or more HpN truncated sequences, homopolymer lengths of the second plurality of DNA molecules may be determined. Such determination may be based at least on (i) the one or more HpN truncated sequences aligned to the HpN truncated references and/or sequence signals associated with the one or more HpN truncated sequences aligned to the HpN truncated references, (ii) the quantified context dependency, or (iii) a combination thereof.

In some embodiments, the quantified context dependency is classified for a given context. Such a given context may be an n-base context, wherein ‘n’ is an integer greater than or equal to 2, an integer greater than or equal to 3, an integer greater than or equal to 4, an integer greater than or equal to 5, an integer greater than or equal to 6, an integer greater than or equal to 7, an integer greater than or equal to 8, an integer greater than or equal to 9, an integer greater than or equal to 10, an integer greater than or equal to 11, an integer greater than or equal to 12, an integer greater than or equal to 13, an integer greater than or equal to 14, an integer greater than or equal to 15, an integer greater than or equal to 16, an integer greater than or equal to 17, an integer greater than or equal to 18, an integer greater than or equal to 19, or an integer greater than or equal to 20.

For example, the quantified context dependency may be classified for an n-base context, in which preliminary sequence calls (e.g., imputed sequences) are grouped by an n-base context (e.g., “tgttca”). The associated signals of the imputed sequences grouped by the n-base context are then used to establish a systematic context mapping. For example, representative signal measurements (signal levels) and signals variations thereof for the individual bases and homopolymers of the imputed sequences within the context (e.g., “t,”, “g,” “tt,” “c,” and “a,” respectively) are measured and recorded as historical data. The historical data may be stored in one or more databases, individually or collectively. A database may comprise any data structure, such as a chart, table, list, array, graph, index, hash database, one or more graphics, or any other type of structure.

As another example, the quantified context dependency may be classified for an n-base context, in which HpN truncated sequences are grouped by a n-base context (e.g., “tgttca”). The associated signals of the HpN truncated sequences grouped by the n-base context are then used to establish a systematic context mapping. For example, representative signal measurements (signal levels) and signals variations thereof for the individual bases and homopolymers of the HpN truncated sequences within the context (e.g., “t,”, “g,” “tt,” “c,” and “a,” respectively) are measured and recorded as historical data (e.g., in a database of systems described herein).

In some embodiments, a context map is generated, which includes a mathematical relationship between a signal and the number of consecutive nucleotides incorporated (e.g., homopolymer length) in a sequence. Such a relationship may be represented as a context specific mapping (context map). A comparison of the true sequences (which comprise homopolymers ranging in length from 2 to 4) and the associated context dependent signals of the true sequences may indicate that there is not a perfectly linear relationship between a homopolymer's signal measurement (signal level) and the homopolymer's length, owing to context dependencies. This non-linear relationship can result in errors in imputed homopolymer lengths, which can then be corrected using historical data and context maps. The monotonic context (e.g., strictly increasing signal by homopolymer length) can be used to map each of a series of signals to correct homopolymer lengths. The context map may be used to train one or more algorithms (e.g., machine learning algorithms) to translate signals to predicted sequences and/or homopolymer lengths. For example, each local context that is found in an imputed sequence may be compared to an aggregated database to retrieve rules that can be applied for the translation.

In some embodiments, the DNA molecules are derived from ribonucleic acid (RNA) molecules. For example, the DNA molecules may be generated by performing reverse transcription on RNA molecules to generate complementary DNA (cDNA) molecules or derivatives thereof. The plurality of sequence signals and/or imputed sequences may be generated by any suitable sequencing approach, such as massively parallel array sequencing, flow sequencing, sequencing by synthesis, or dye sequencing. In some embodiments, quantifying the context dependency comprises establishing a relationship between signal amplitudes and homopolymer length for each of a plurality of loci. Such a relationship may be represented as a context specific mapping (context map).

Methods of the present disclosure may comprise quantifying context dependency of a plurality of sequence signals and imputed sequences. Such a method may comprise sequencing deoxyribonucleic acid (DNA) molecules to provide a plurality of sequence signals and imputed sequences. In some cases, the DNA molecules comprise a known sequence. From such imputed sequences, homopolymer sequences (e.g., a sequence containing a homopolymer comprising multiple consecutive nucleotides of the same base) of at least N bases may be identified. These identified imputed homopolymer sequences may then be truncated to a homopolymer sequence of bases of length N, to yield one or more HpN truncated sequences. The length N may be any number of a plurality of bases, such as 2 bases, 3 bases, 4 bases, 5 bases, 6 bases, 7 bases, 8 bases, 9 bases, 10 bases, 11 bases, 12 bases, 13 bases, 14 bases, 15 bases, or more than 15 bases. After truncation, the one or more HpN truncated sequences may be aligned to one or more truncated references. Such truncated references may be HpN truncated and thereby comprise one or more homopolymer sequences truncated to length N. After alignment of the one or more HpN truncated sequences, an expected signal for each of a plurality of loci in the HpN truncated references may be determined. Such expected signal may be determined based at least on (i) the one or more HpN truncated sequences aligned to the HpN truncated references and/or sequence signals associated with the one or more HpN truncated sequences aligned to the HpN truncated reference(s), (ii) the known sequence, or (iii) a combination thereof.

In some embodiments, quantifying context dependency of a plurality of sequence signals and imputed sequences comprises sequencing a second set of DNA molecules comprising unknown sequences, thereby generating a second plurality of sequence signals and imputed sequences. From such imputed sequences, second homopolymer sequences (e.g., a sequence containing a homopolymer comprising multiple consecutive nucleotides of the same base) of at least N bases may be identified. These identified imputed second homopolymer sequences may then be truncated to a homopolymer sequence of bases of length N, to yield one or more second HpN truncated sequences. The length N may be any number of a plurality of bases, such as 2 bases, 3 bases, 4 bases, 5 bases, 6 bases, 7 bases, 8 bases, 9 bases, 10 bases, 11 bases, 12 bases, 13 bases, 14 bases, 15 bases, or more than 15 bases. After truncation, the one or more second HpN truncated sequences may be aligned to the one or more HpN truncated references. After alignment of the one or more HpN truncated sequences, homopolymer lengths of the second plurality of DNA molecules may be determined. Such determination may be based at least on (i) the one or more HpN truncated sequences aligned to the HpN truncated references and/or sequence signals associated with the one or more HpN truncated sequences aligned to the HpN truncated references, (ii) the quantified context dependency, or (iii) a combination thereof.

In some embodiments, the DNA molecules are derived from ribonucleic acid (RNA) molecules. For example, the DNA molecules may be generated by performing reverse transcription on RNA molecules to generate complementary DNA (cDNA) molecules or derivatives thereof. The plurality of sequence signals and/or imputed sequences may be generated by any suitable sequencing approach, such as massively parallel array sequencing, flow sequencing, sequencing by synthesis, or dye sequencing. In some embodiments, quantifying the context dependency comprises establishing a relationship between signal amplitudes and homopolymer length for each of a plurality of loci. Such a relationship may be represented as a context specific mapping (context map).

Methods of the present disclosure may comprise processing a plurality of sequence signals. Such a method may be used to determine homopolymer lengths by incorporation of secondary assay data. The method may comprise sequencing a nucleic acid sample to provide a plurality of sequence signals and imputed sequences. The plurality of sequence signals and imputed sequences may be processed to determine a set of one or more sequences comprising homopolymer sequences. The plurality of sequence signals and imputed sequences may also be processed to identify a presence and/or an estimated length of at least a portion of the homopolymer sequences. One or more algorithms may be used to identify the presence and/or the estimated length of the homopolymer sequences, by translating signals to homopolymer lengths (e.g., using a context map or other context dependency information). The estimated lengths of the homopolymer sequences may be refined using secondary assay data. Such secondary assay data may be used to provide or augment context dependency information. The plurality of sequence signals and/or imputed sequences may be generated by any suitable sequencing approach, such as massively parallel array sequencing, flow sequencing, sequencing by synthesis, or dye sequencing.

### Methods for Analog Alignment

Methods of the present disclosure may comprise processing a plurality of sequence signals, to determine base calls by alignment of a signal to a reference signal (e.g., an analog reference signal). The method may comprise sequencing a nucleic acid sample to provide the plurality of sequence signals. The plurality of sequence signals may be aligned to a reference signal (e.g., an analog reference signal). Based at least on the aligned sequence signals, a reference locus comprising a sequence of bases may be identified. A consensus sequence may be generated from the plurality of sequence signals aligned to the reference signal. The consensus sequence may comprise a sequence of N bases. The generation may be performed based at least on the identified reference locus, a length of the sequence of the reference locus, and the reference signal (e.g., analog reference signal).

In some embodiments, the method for processing a plurality of sequence signals may comprise calculating a length estimation error of the sequence. The length estimation error may comprise a confidence interval for the length of the sequence. For example, the length estimation error for a sequence with an imputed length of 5 bases may comprise a confidence interval of [3, 7], or 5 bases ±2 bases. The length estimation error may be calculated based at least on a distribution of signals or imputed sequence lengths of the plurality of sequence signals aligned to the reference signal.

In some embodiments, processing a plurality of sequence signals may comprise pre-processing the plurality of sequence signals to remove systematic errors. Such pre-processing may be performed prior to aligning the plurality of sequence signals to the reference signal. The pre-processing may be performed to address random and unpredictable systematic variations in signal level, which can cause errors in base calling the sequence. In some cases, instrument and detection systematic variation can be calibrated and removed by monitoring instrument diagnostics and common-mode behavior across large numbers of colonies.

In some embodiments, the plurality of sequence signals is generated by sequencing nucleic acids of a subject. In some cases, a number of lengths computed or classified when generating the consensus sequence may be restricted, based at least on the ploidy of the species of the subject. The plurality of sequence signals may be generated by any suitable sequencing approach, such as massively parallel array sequencing, flow sequencing, sequencing by synthesis, or dye sequencing.

Methods of the present disclosure may comprise quantifying context dependency of a plurality of sequence signals. The method may comprise sequencing deoxyribonucleic acid (DNA) or ribonucleic acid (RNA) molecules to provide the plurality of sequence signals. The DNA or RNA molecules may comprise a known sequence. The plurality of sequence signals may be aligned to a reference signal (e.g., an analog reference signal). The context dependency may be quantified in the plurality of sequence signals aligned to the reference signal. The quantification of context dependency may be performed based at least on the known sequence. In some embodiments, the aligning may comprise performing one or more analog signal processing algorithms.

In some embodiments, quantifying context dependency of a plurality of sequence signals comprises sequencing a second set of DNA molecules comprising unknown sequences, thereby generating a second plurality of sequence signals. The second plurality of sequence signals may be aligned to the reference signal (e.g., analog reference signal). After alignment of the second plurality of sequence signals, base calls of the second plurality of DNA molecules may be determined. Such determination may be based at least on the plurality of sequence signals aligned to the reference signal, the quantified context dependency, or a combination thereof.

In some embodiments, the DNA molecules are derived from ribonucleic acid (RNA) molecules. For example, the DNA molecules may be generated by performing reverse transcription on RNA molecules to generate complementary DNA (cDNA) molecules or derivatives thereof. The plurality of sequence signals and/or imputed sequences may be generated by any suitable sequencing approach, such as massively parallel array sequencing, flow sequencing, sequencing by synthesis, or dye sequencing. In some embodiments, quantifying the context dependency comprises establishing a relationship between signal amplitudes and base calls and/or sequence length for each of a plurality of loci. Such a relationship may be represented as a context specific mapping (context map).

Methods of the present disclosure may comprise quantifying context dependency of a plurality of sequence signals. The method may comprise sequencing deoxyribonucleic acid (DNA) or ribonucleic acid (RNA) molecules to provide the plurality of sequence signals. The DNA or RNA molecules may comprise a known sequence. The plurality of sequence signals may be aligned to a reference signal (e.g., an analog reference signal). After alignment of the plurality of sequence signals to a reference signal, an expected signal may be determined for each of a plurality of loci in the reference signal. The determination may be performed based at least on the plurality of sequence signals aligned to the reference signal, the known sequence, or a combination thereof. In some embodiments, the aligning may comprise performing one or more analog signal processing algorithms.

In some embodiments, quantifying context dependency of a plurality of sequence signals comprises sequencing a second set of DNA molecules comprising unknown sequences, thereby generating a second plurality of sequence signals. The second plurality of sequence signals may be aligned to the reference signal (e.g., analog reference signal). After alignment of the second plurality of sequence signals, base calls of the second plurality of DNA molecules may be determined. Such determination may be based at least on the plurality of sequence signals aligned to the reference signal, the quantified context dependency, or a combination thereof.

In some embodiments, the DNA molecules are derived from ribonucleic acid (RNA) molecules. For example, the DNA molecules may be generated by performing reverse transcription on RNA molecules to generate complementary DNA (cDNA) molecules or derivatives thereof. The plurality of sequence signals and/or imputed sequences may be generated by any suitable sequencing approach, such as massively parallel array sequencing, flow sequencing, sequencing by synthesis, or dye sequencing. In some embodiments, quantifying the context dependency comprises establishing a relationship between signal amplitudes and base calls and/or sequence length for each of a plurality of loci. Such a relationship may be represented as a context specific mapping (context map).

Methods of the present disclosure may comprise processing a plurality of sequence signals. The method may comprise sequencing a nucleic acid sample to provide the plurality of sequence signals. The plurality of sequence signals may be aligned to a reference signal (e.g., an analog reference signal). After aligning the plurality of sequence signals to a reference signal, a genomic locus comprising a sequence of bases may be identified. The identification may be performed based at least on the aligned sequence signals. The plurality of sequence signals aligned to the reference signal may be processed to identify base calls and/or an estimated length of the sequence of bases. One or more algorithms may be used to identify the base calls and/or the estimated length of the sequence of bases, by translating signals to base calls and sequence lengths (e.g., using a context map or other context dependency information). The estimated base calls and sequence lengths of the sequences may be refined using secondary assay data. Such secondary assay data may be used to provide or augment context dependency information. The plurality of sequence signals may be generated by any suitable sequencing approach, such as massively parallel array sequencing, flow sequencing, sequencing by synthesis, or dye sequencing.

### Computer Systems

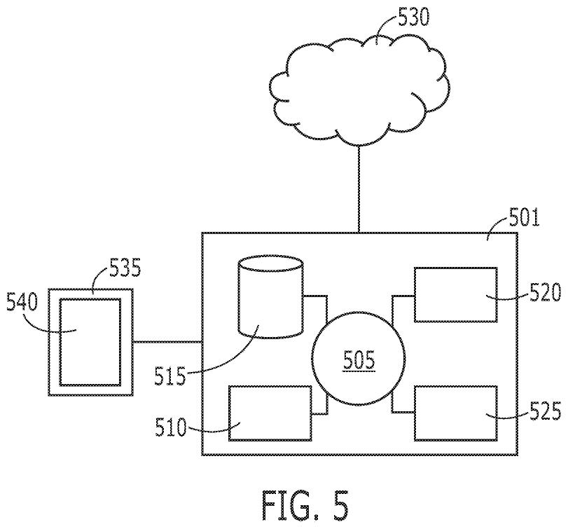

The present disclosure provides computer control systems that are programmed to implement methods of the disclosure. FIG. 5 shows a computer system 501 that is programmed or otherwise configured to, for example: generate sets of barcodes for use in barcoding nucleic acid molecules; sequence barcoded nucleic acid molecules to generate sequencing signals comprising signals corresponding to the barcode sequences; and/or use the signals corresponding to the barcode sequences to group the sequencing signals into groups, wherein sequencing signals of a given group comprise signals corresponding to a barcode sequence that is (i) identical for the given group and (ii) different from barcode sequences of other groups; process the sequencing signals within the given group to generate sets of aggregated signals; and combine the sets of aggregated signals to generate a consensus sequence.

The computer system 501 can regulate various aspects of methods and systems of the present disclosure, such as, for example, generating sets of barcodes for use in barcoding nucleic acid molecules; sequencing barcoded nucleic acid molecules to generate sequencing signals comprising signals corresponding to the barcode sequences; using the signals corresponding to the barcode sequences to group the sequencing signals into groups, wherein sequencing signals of a given group comprise signals corresponding to a barcode sequence that is (i) identical for the given group and (ii) different from barcode sequences of other groups; processing the sequencing signals within the given group to generate sets of aggregated signals; and combining the sets of aggregated signals to generate a consensus sequence.

The computer system 501 can be an electronic device of a user or a computer system that is remotely located with respect to the electronic device. The electronic device can be a mobile electronic device. The computer system 501 includes a central processing unit (CPU, also “processor” and “computer processor” herein) 505, which can be a single core or multi core processor, or a plurality of processors for parallel processing. The computer system 501 also includes memory or memory location 510 (e.g., random-access memory, read-only memory, flash memory), electronic storage unit 515 (e.g., hard disk), communication interface 520 (e.g., network adapter) for communicating with one or more other systems, and peripheral devices 525, such as cache, other memory, data storage and/or electronic display adapters. The memory 510, storage unit 515, interface 520 and peripheral devices 525 are in communication with the CPU 505 through a communication bus (solid lines), such as a motherboard. The storage unit 515 can be a data storage unit (or data repository) for storing data. The computer system 501 can be operatively coupled to a computer network (“network”) 530 with the aid of the communication interface 520. The network 530 can be the Internet, an internet and/or extranet, or an intranet and/or extranet that is in communication with the Internet. The network 530 in some cases is a telecommunication and/or data network. The network 530 can include one or more computer servers, which can enable distributed computing, such as cloud computing. The network 530, in some cases with the aid of the computer system 501, can implement a peer-to-peer network, which may enable devices coupled to the computer system 501 to behave as a client or a server.

The CPU 505 can execute a sequence of machine-readable instructions, which can be embodied in a program or software. The instructions may be stored in a memory location, such as the memory 510. The instructions can be directed to the CPU 505, which can subsequently program or otherwise configure the CPU 505 to implement methods of the present disclosure. Examples of operations performed by the CPU 505 can include fetch, decode, execute, and writeback.

The CPU 505 can be part of a circuit, such as an integrated circuit. One or more other components of the system 501 can be included in the circuit. In some cases, the circuit is an application specific integrated circuit (ASIC).

The storage unit 515 can store files, such as drivers, libraries and saved programs. The storage unit 515 can store user data, e.g., user preferences and user programs. The computer system 501 in some cases can include one or more additional data storage units that are external to the computer system 501, such as located on a remote server that is in communication with the computer system 501 through an intranet or the Internet.

The computer system 501 can communicate with one or more remote computer systems through the network 530. For instance, the computer system 501 can communicate with a remote computer system of a user. Examples of remote computer systems include personal computers (e.g., portable PC), slate or tablet PC's (e.g., Apple® iPad, Samsung® Galaxy Tab), telephones, Smart phones (e.g., Apple® iPhone, Android-enabled device, Blackberry®), or personal digital assistants. The user can access the computer system 501 via the network 530.

Methods as described herein can be implemented by way of machine (e.g., computer processor) executable code stored on an electronic storage location of the computer system 501, such as, for example, on the memory 510 or electronic storage unit 515. The machine executable or machine readable code can be provided in the form of software. During use, the code can be executed by the processor 505. In some cases, the code can be retrieved from the storage unit 515 and stored on the memory 510 for ready access by the processor 505. In some situations, the electronic storage unit 515 can be precluded, and machine-executable instructions are stored on memory 510.

The code can be pre-compiled and configured for use with a machine having a processer adapted to execute the code, or can be compiled during runtime. The code can be supplied in a programming language that can be selected to enable the code to execute in a pre-compiled or as-compiled fashion.

Aspects of the systems and methods provided herein, such as the computer system 501, can be embodied in programming. Various aspects of the technology may be thought of as “products” or “articles of manufacture” typically in the form of machine (or processor) executable code and/or associated data that is carried on or embodied in a type of machine readable medium. Machine-executable code can be stored on an electronic storage unit, such as memory (e.g., read-only memory, random-access memory, flash memory) or a hard disk. “Storage” type media can include any or all of the tangible memory of the computers, processors or the like, or associated modules thereof, such as various semiconductor memories, tape drives, disk drives and the like, which may provide non-transitory storage at any time for the software programming. All or portions of the software may at times be communicated through the Internet or various other telecommunication networks. Such communications, for example, may enable loading of the software from one computer or processor into another, for example, from a management server or host computer into the computer platform of an application server. Thus, another type of media that may bear the software elements includes optical, electrical and electromagnetic waves, such as used across physical interfaces between local devices, through wired and optical landline networks and over various air-links. The physical elements that carry such waves, such as wired or wireless links, optical links or the like, also may be considered as media bearing the software. As used herein, unless restricted to non-transitory, tangible “storage” media, terms such as computer or machine “readable medium” refer to any medium that participates in providing instructions to a processor for execution.

Hence, a machine readable medium, such as computer-executable code, may take many forms, including but not limited to, a tangible storage medium, a carrier wave medium or physical transmission medium. Non-volatile storage media include, for example, optical or magnetic disks, such as any of the storage devices in any computer(s) or the like, such as may be used to implement the databases, etc. shown in the drawings. Volatile storage media include dynamic memory, such as main memory of such a computer platform. Tangible transmission media include coaxial cables; copper wire and fiber optics, including the wires that comprise a bus within a computer system. Carrier-wave transmission media may take the form of electric or electromagnetic signals, or acoustic or light waves such as those generated during radio frequency (RF) and infrared (IR) data communications. Common forms of computer-readable media therefore include for example: a floppy disk, a flexible disk, hard disk, magnetic tape, any other magnetic medium, a CD-ROM, DVD or DVD-ROM, any other optical medium, punch cards paper tape, any other physical storage medium with patterns of holes, a RAM, a ROM, a PROM and EPROM, a FLASH-EPROM, any other memory chip or cartridge, a carrier wave transporting data or instructions, cables or links transporting such a carrier wave, or any other medium from which a computer may read programming code and/or data. Many of these forms of computer readable media may be involved in carrying one or more sequences of one or more instructions to a processor for execution.

The computer system 501 can include or be in communication with an electronic display 535 that comprises a user interface (UI) 540 for providing, for example, user selection of algorithms, signal data, sequence data, and databases. Examples of UI's include, without limitation, a graphical user interface (GUI) and web-based user interface.

Methods and systems of the present disclosure can be implemented by way of one or more algorithms. An algorithm can be implemented by way of software upon execution by the central processing unit 505. The algorithm can, for example, generate sets of barcodes for use in barcoding nucleic acid molecules; sequence barcoded nucleic acid molecules to generate sequencing signals comprising signals corresponding to the barcode sequences; use the signals corresponding to the barcode sequences to group the sequencing signals into groups, wherein sequencing signals of a given group comprise signals corresponding to a barcode sequence that is (i) identical for the given group and (ii) different from barcode sequences of other groups; process the sequencing signals within the given group to generate sets of aggregated signals; and combine the sets of aggregated signals to generate a consensus sequence.

### Integrating Sequencing Signals for Accurate Base Calling

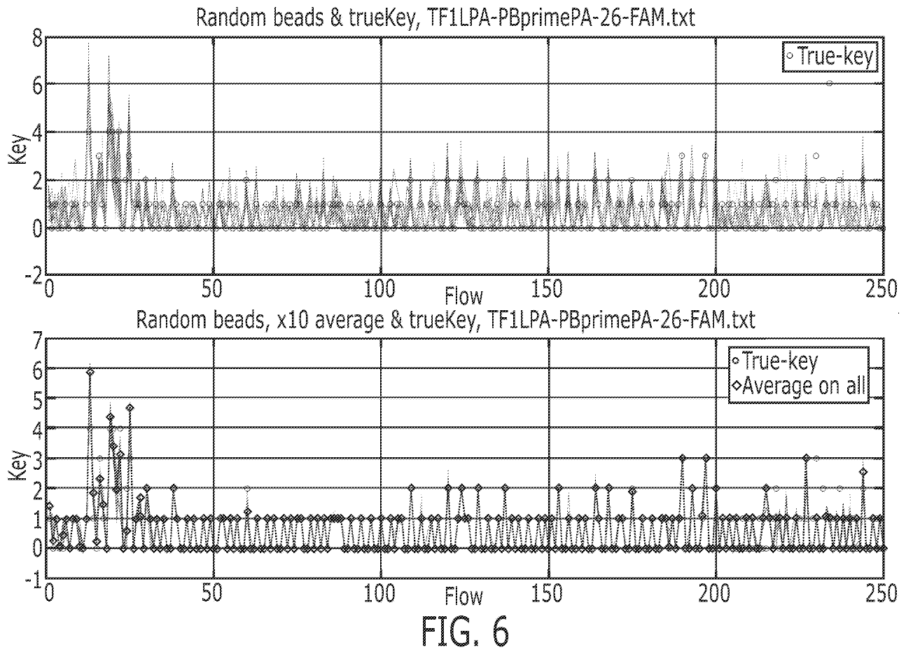

As depicted in FIG. 1, raw sequencing signals (e.g., fluorescent measurements during each flow cycle) can be used as a basis for accurately grouping sequencing data. In particular, the raw signals provide the possibility of using analytic methods, such as signal averaging, to reduce or eliminate systematic errors. As a result, sorting based on raw signals can be more accurate. As illustration, examples are presented in FIGS. 6-9. Data averaging techniques may be applied to raw sequencing data, leading to more accurate base calling across multiple template molecules. Similar results are observed when different neural network models are used for base calling.

In some embodiments, averaging techniques can be applied at different stages of the analysis, to raw signals (where number of raw signals to be averaged can vary by, for example, 10-fold, 100-fold, 1000-fold, 10,000-fold, or greater). The averaged signals may then be used as inputs to a trained model for base calling (e.g., a human-genome trained neural network model or an E. coli-genome trained neural network model). In some embodiments, raw signals can still be supplied to a trained model for base calling but outputs from the base calling model can be averaged. For example, the trained model can output a number of probabilities (e.g., 4 probabilities) each corresponding to the likelihood of a particular base type being presenting at a given position based on data from a bead hybridized to a particular template. Output probabilities calculated from multiple beads hybridized to the same template can then be averaged. In some embodiments, averaging techniques can be applied at multiple levels. For example, raw signals can be averaged for every ten beads hybridized to the same template molecule and the averaged data are used as input to a trained model for base calling, and additionally output from the base calling model can be averaged across different groups of ten beads (e.g., each ten beads can be treated as a super bead).

Even though the analysis described may be performed in connection with template molecules, similar approaches can be performed in connection with the barcode sequence or signal grouping and subgrouping analysis (e.g., as outlined in FIG. 1). For example, each of the template molecule in the examples below (or a portion thereof) can be considered as a barcode. Applying the methods disclosed herein may lead to more accurate grouping based on barcode sequence. Additionally, if a portion of a template molecule is treated as a barcode, the remainder of the template molecule sequence can also be considered as a target molecule (e.g., one subject to variant analysis). More accurate barcode group in combination with more accurate base calling in the target region can improve accuracy of variant identification.

### EXAMPLES

### Example 1

Using methods and systems of the present disclosure, sequencing data of several known templates was used to demonstrate the advantageous effect of performing improved base calling via a plurality of averaging techniques (e.g., averaging sequencing signals thereby creating a “hyper-bead,” averaging output from a base caller algorithm prior to base calling, through a combination of averaging techniques, etc.). Such analyses may be performed without using molecular barcodes to distinguish between individual template molecules from among a plurality of template molecules. The performance analysis comprised comparing, for each of a plurality of template molecules, the error rate of base calling performed on a hyper-bead associated with the plurality of template molecules (e.g., using one or more averaging techniques) as compared to the error rate of base calling performed based on input from a plurality of beads associated with the plurality of template molecules (e.g., without averaging).

In some embodiments, a template molecule was chosen (e.g., from among TF1L, TF2L, TF3L, TF4L, TF5L, TF6L, etc.) for a particular experiment. Next, sequencing data were collected for the template molecule; for example, from a plurality of beads each bearing the template molecule. Next, using a neural network model (e.g., trained on the human genome, an E. coli genome, or another reference genome), base calling was performed on the plurality of individual template reads from each bead hybridized to the same template molecule, thereby determining the sequence information of the template molecule. Next, an error rate per template was determined across multiple beads that were included in the analysis (e.g., using a single run).

In some embodiments, for a given template type, the signals for a plurality of beads for the given template type were averaged together to create a “hyper-bead.” For example, a “hyper-bead” can be generated by averaging signals from about 5 beads, about 10 beads, about 20 beads, about 30 beads, about 40 beads, about 50 beads, about 60 beads, about 70 beads, about 80 beads, about 90 beads, about 100 beads, about 200 beads, about 300 beads, about 400 beads, about 500 beads, about 600 beads, about 700 beads, about 800 beads, about 900 beads, about 1000 beads, about 2000 beads, about 3000 beads, about 4000 beads, about 5000 beads, about 6000 beads, about 7000 beads, about 8000 beads, about 9000 beads, about 10000 beads, etc. Next, using the same human-genome trained neural network model, base calling was performed on the hyper-bead. Next, an error rate for the hyper-bead was determined and compared to the error rate per template, thereby confirming that the error rate is reduced by the signal averaging technique of the base calling using hyper-beads.

In some embodiments, after confirming that the signal averaging technique results in demonstrated performance improvement over all beads, the experiment is repeated for a given template molecule for a smaller plurality of beads (e.g., by averaging signals across groups of about 5 beads, about 10 beads, about 20 beads, about 30 beads, about 40 beads, about 50 beads, about 60 beads, about 70 beads, about 80 beads, about 90 beads, about 100 beads, about 200 beads, about 300 beads, about 400 beads, about 500 beads, about 600 beads, about 700 beads, about 800 beads, about 900 beads, about 1000 beads, about 2000 beads, about 3000 beads, about 4000 beads, about 5000 beads, about 6000 beads, about 7000 beads, about 8000 beads, about 9000 beads, about 10000 beads, etc.).

When another template molecule is chosen, the experiment can be repeated with the different template molecule.

The experiments were performed on each of a plurality of 6 standard template molecules TF1L, TF2L, TF3L, TF4L, TF5L, and TF6L. Further, base calling experiments were performed using two separately trained neural network models: a first neural network model trained on the human genome (the human or HG NN model) and a second neural network trained on the E. coli genome (the E. coli NN model).

FIG. 6 shows an example of base call analysis of a TF1L template. Here, florescent signals were quantified for each flow cycle during which a specific type of nucleotide was made accessible to the extending template molecule. Base calling was performed using a human genome-trained neural network model. The top panel illustrates base calling results from randomly selected beads each hybridized to a TF1L template without signal averaging. True-key indicating the actual template sequence is shown as dark circles. Base call results from individual beads are depicted without specifying base type for simplicity. As shown in the figure, base call results from different beads scatter across each cycle with considerable fluctuation. The bottom panel illustrates base calling results using a signal averaging technique; e.g., based on 100 average signals, each measured across randomly selected pluralities of 10 beads each hybridized to a TF1L template. An “average on all” plot depicts the neural network prediction once signals are averaged across a large number of beads (e.g., a few tens of thousands of beads). Alternatively, averages can be calculated based on output from the neural network models. Still alternatively, a combined averaging method can be used. For example, florescent signals can be averaged for each group of beads (e.g., each group contains 10 to 100 beads). The averaged signals are then used as input to a pre-trained neural network model for base calling. The output from the neural network model (e.g., probability values each representing a likelihood that a particular base type is present at a particular position in the template) can be further averaged before a final base call for the particular position.

The top panel reveals that, without averaging, signals from randomly selected beads scatter around and sometimes deviate significantly from the true key base type. In contrast, average signals consistently lead to accurate base calls that agree with those in the true key.

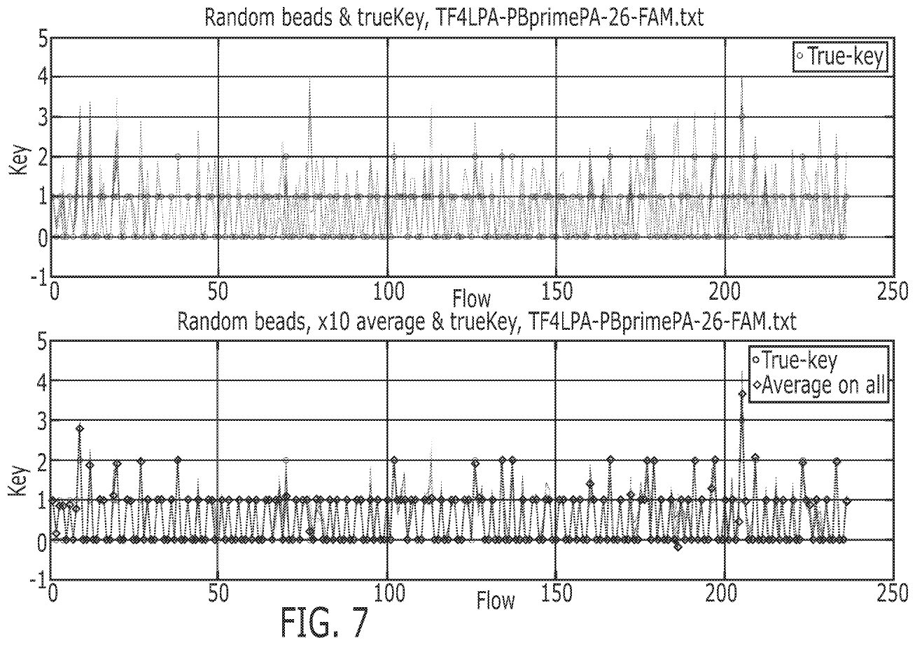

FIG. 7 shows an example of base call analysis of a TF4L template. Here, florescent signals were quantified for each flow cycle during which a specific type of nucleotide was made accessible to the extending template molecule. Base calling was performed using a human genome-trained neural network model and data are presented in manner similar to those in FIG. 6. Similar results were observed. The top panel of FIG. 7 also reveals that, without averaging, signals from randomly selected beads scatter around and sometimes deviate significantly from the true key base type. In contrast, average signals consistently lead to accurate base calls that agree with those in the true key.

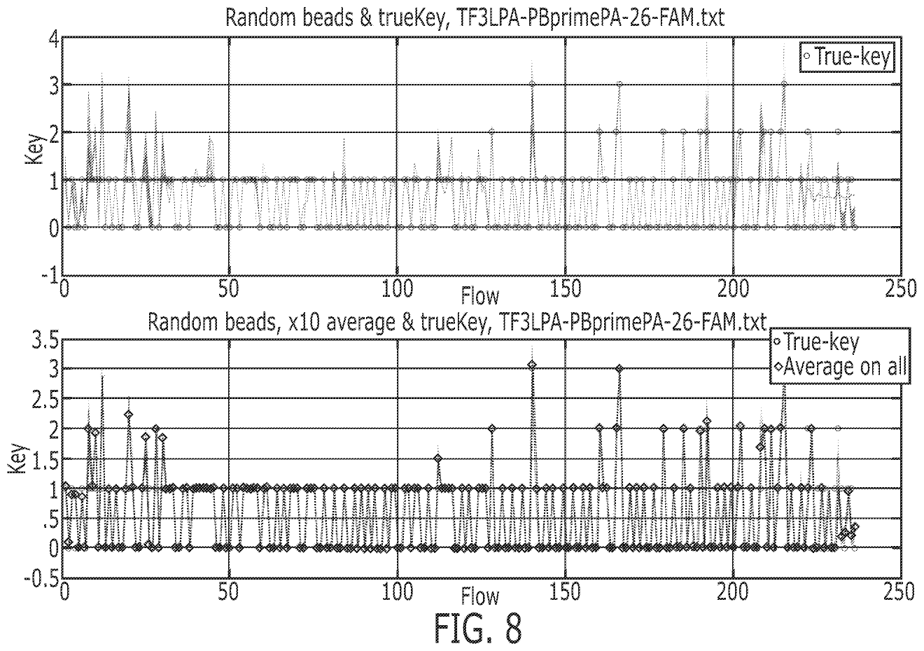

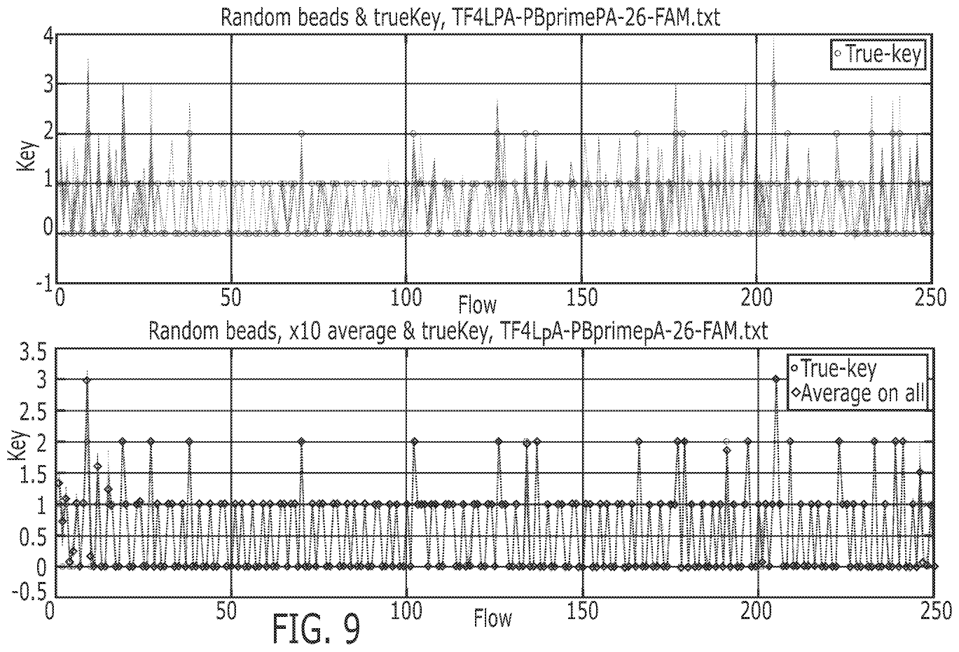

FIG. 8 shows an example of base call analysis of a TF3L template, using an E. coli genome-trained neural network model for base calling. FIG. 9 shows an example of base call analysis of a TF4L template using an E. coli genome-trained neural network model for base calling. Results similar to those observed using a pre-trained human neural network model were observed in the two experiments depicted in FIGS. 8-9. Without averaging, signals from randomly selected beads scatter around and sometimes deviate significantly from the true key base type. In contrast, average signals consistently lead to accurate base calls that agree with those in the true key.

Table 1 shows a summary of bead error rates (BER) obtained for various bead calling experiments using different template molecules (e.g., PhiX-2941L, TF1L, TF3L, TF4L, TF5L, and TF6L) and using different neural network models (e.g., a human NN model and an E. coli NN model).

TABLE 1
Bead error rates across template molecules using human and
E. coli NN models

| | | | Averag- | Averag- | Averag- | Error |
|---|---|---|---|---|---|---|
| | Error | Averag- | ing | ing | ing | average |
| | for all | ing | 100 | 1000 | all | reg- |
| | reads | 10 beads | beads | beads | beads | signal |
| Template | (%) | (%) | (%) | (%) | (%) | (%) |
| PhiX- | 2.0493 | 0.0092663 | 0 | 0 | 0 | 0 |
| 2941L | | | | | | |
| (Human | | | | | | |
| NN | | | | | | |
| model) | | | | | | |
| PhiX- | 2.6802 | 1.0986 | 1.0947 | 0.93458 | 0.93458 | |
| 2941L | | | | | | |
| (E. coli | | | | | | |
| NN | | | | | | |
| model) | | | | | | |
| TF1L | 12.129 | 1.1232 | 1.0659 | 1.0989 | 1.0989 | 1.0989 |
| (Human | | | | | | |
| NN | | | | | | |
| model) | | | | | | |
| TF1L | 1.0842 | 0.032163 | 0 | 0 | 0 | |
| (E. coli | | | | | | |
| NN | | | | | | |
| model) | | | | | | |
| TF5L | 1.436 | 0.0015893 | 0 | 0 | 0 | 0 |
| (Human | | | | | | |
| NN | | | | | | |
| model) | | | | | | |
| TF5L | 0.86247 | 0.60626 | 0.83941 | 1.0309 | 1.0309 | |
| (E. coli | | | | | | |
| NN | | | | | | |
| model) | | | | | | |
| TF6L | 12.7359 | 9.4995 | 9.6676 | 9.8862 | 9.9099 | 9.009 |
| (Human | | | | | | |
| NN | | | | | | |
| model) | | | | | | |
| TF6L | 10.0564 | 9.031 | 9.009 | 9.009 | 9.009 | |
| (E. coli | | | | | | |
| NN | | | | | | |
| model) | | | | | | |
| TF3L | 1.311 | 0.046695 | 0 | 0 | 0 | 0 |
| (Human | | | | | | |
| NN | | | | | | |
| model) | | | | | | |
| TF3L | 1.8309 | 0.65894 | 0.54361 | 0.401 | 0 | |
| (E. coli | | | | | | |
| NN | | | | | | |
| model) | | | | | | |
| TF4L | 4.2749 | 0.35966 | 0.022579 | 0 | 0 | 0 |
| (Human | | | | | | |
| NN | | | | | | |
| model) | | | | | | |
| TF4L | 15.411 | 3.7176 | 1.5989 | 1.111 | 1.111 | |
| (E. coli | | | | | | |
| NN | | | | | | |
| model) | | | | | | |

As shown in FIGS. 6-9 and Table 1, the results of the experiments across these 6 standard template molecules were reported, including the bead error rate (BER) for the standard 6 templates using various techniques, including base calling with all individual errors per beads, base calling with signal averaging across 10 beads, base calling with signal averaging across 100 beads, base calling with signal averaging across 1000 beads, base calling with signal averaging across all beads. In particular, the results demonstrate that, for most of templates, performing base calling using the signal averaging technique generally reduces the BER (notwithstanding a few cases for which BER was not improved due to systematic errors). Therefore, the data obtained from the experiments clearly demonstrate that in some cases, performing base calling using a signal averaging technique effectively reduces BER as a result of increased signal-to-noise (SNR). Such improvements in SNR are realized by the effective error suppression of “noise” arising from random errors. This improvement in SNR was particularly evident, for example, in templates TF1L, TF3L, and TF4L. Further, the NN model corrects for some of the variability in signals (e.g., cross-wafer variability, and non-linear dependence on copy number), thereby increasing the SNR of base calling.

While preferred embodiments of the present invention have been shown and described herein, it will be obvious to those skilled in the art that such embodiments are provided by way of example only. It is not intended that the invention be limited by the specific examples provided within the specification. While the invention has been described with reference to the aforementioned specification, the descriptions and illustrations of the embodiments herein are not meant to be construed in a limiting sense. Numerous variations, changes, and substitutions will now occur to those skilled in the art without departing from the invention. Furthermore, it shall be understood that all aspects of the invention are not limited to the specific depictions, configurations or relative proportions set forth herein which depend upon a variety of conditions and variables. It should be understood that various alternatives to the embodiments of the invention described herein may be employed in practicing the invention. It is therefore contemplated that the invention shall also cover any such alternatives, modifications, variations or equivalents. It is intended that the following claims define the scope of the invention and that methods and structures within the scope of these claims and their equivalents be covered thereby.

1. A method for sequencing a plurality of nucleic acid molecules, comprising:
(a) using a plurality of barcode molecules to barcode a plurality of nucleic acid molecules from a biological sample, to generate a plurality of barcoded nucleic acid molecules comprising a plurality of barcode sequences;
(b) sequencing said plurality of barcoded nucleic acid molecules or a derivative thereof to generate a plurality of sequencing signals, which plurality of sequencing signals comprises signals corresponding to said plurality of barcode sequences, wherein said plurality of sequencing signals are not sequencing reads;
(c) using said signals corresponding to said plurality of barcode sequences to group said plurality of sequencing signals into a plurality of groups, wherein sequencing signals of a given group of said plurality of groups comprise signals corresponding to a barcode sequence of said plurality of barcode sequences that is (i) identical for said given group and (ii) different from barcode sequences of other groups of said plurality of groups;
(d) processing said sequencing signals within said given group to generate one or more sets of aggregated signals, wherein said one or more sets of aggregated signals are not sequencing reads; and
(e) combining said one or more sets of aggregated signals to generate a consensus sequence.

2. The method of claim 1, wherein in (e), said combining comprises performing base calling to identify individual bases.

3. The method of claim 2, wherein said base calling is performed by processing aggregated signals within each of said one or more sets of aggregated signals to each other to generate said consensus sequence.

4. The method of claim 3, further comprising averaging said aggregated signals within each of said one or more sets of aggregated signals to each other to generate said consensus sequence.

5. The method of claim 3, further comprising processing said consensus sequence against a reference to identify one or more genetic variants.

6. The method of claim 2, wherein said base calling is performed by processing aggregated signals within each of said one or more sets of aggregated signals against a reference signal to generate said consensus sequence.

7. (canceled)

8. The method of claim 1, wherein said plurality of nucleic acid molecules comprises deoxyribonucleic acid (DNA) molecules or ribonucleic acid molecules (RNA).

9. The method of claim 8, wherein said plurality of nucleic acid molecules comprises methylated DNA molecules.

10. (canceled)

11. The method of claim 1, wherein in (a), said barcoding comprises ligating said barcode molecules to said plurality of nucleic acid molecules.

12. The method of claim 1, wherein said plurality of barcoded nucleic acid molecules is non-uniquely barcoded.

13. The method of claim 1, wherein said plurality of barcode molecules comprises at least about 100,000 distinct barcodes.

14. The method of claim 1, wherein said plurality of barcode molecules comprises a Hamming distance of at least 2 nucleotide substitutions.

15. The method of claim 1, wherein said plurality of sequencing signals comprises analog signals.

16. The method of claim 1, further comprising, prior to or after (c), pre-processing said plurality of sequencing signals to remove systematic errors.

17. The method of claim 1, further comprising, prior to (b), amplifying said plurality of barcoded nucleic acid molecules.

18. The method of claim 17, wherein said amplifying comprises polymerase chain reaction (PCR) or recombinase polymerase amplification (RPA).

19. (canceled)

20. The method of claim 1, wherein said plurality of sequencing signals is generated by massively parallel array sequencing.

21. The method of claim 1, wherein said plurality of sequencing signals is generated by flow sequencing.

22. The method of claim 1, wherein (c) and (d) are performed in real time or near real time with said sequencing of (b).

23. The method of claim 22, wherein (e) is performed in real time or near real time with said sequencing of (b).

24-90. (canceled)
